SEQUENCE LISTING (19 sequences)

SEQ ID NO: 1 - DNA, 20 bp, Artificial Sequence
  Description of Artificial Sequence Synthetic primer
gtgccctacatcatcgacct

SEQ ID NO: 2 - DNA, 20 bp, Artificial Sequence
  Description of Artificial Sequence Synthetic primer
ctgcgacatgctgttgaagt

SEQ ID NO: 3 - DNA, 26 bp, Artificial Sequence
  Description of Artificial Sequence Synthetic primer
gatgccaacatccaggacaacatggg

SEQ ID NO: 4 - DNA, 26 bp, Artificial Sequence
  Description of Artificial Sequence Synthetic primer
ggcaggcggtccatatgatccgtgat

SEQ ID NO: 5 - DNA, 20 bp, Artificial Sequence
  Description of Artificial Sequence Synthetic primer
acatcatcacagacttggtc

SEQ ID NO: 6 - DNA, 20 bp, Artificial Sequence
  Description of Artificial Sequence Synthetic primer
cattattgacagcagctgcc

SEQ ID NO: 7 - DNA, 24 bp, Artificial Sequence
  Description of Artificial Sequence Synthetic primer
acacacctgaaggggtgcggtata

SEQ ID NO: 8 - DNA, 25 bp, Artificial Sequence
  Description of Artificial Sequence Synthetic primer
agggctgcagtcattggtattctga

SEQ ID NO: 9 - DNA, 25 bp, Artificial Sequence
  Description of Artificial Sequence Synthetic primer
cagtggctttactggcacctactgc

SEQ ID NO: 10 - DNA, 25 bp, Artificial Sequence
  Description of Artificial Sequence Synthetic primer
gggttgcagtcgttggtattgtgag

SEQ ID NO: 11 - DNA, 23 bp, Artificial Sequence
  Description of Artificial Sequence Synthetic primer
tgctgggcgtcgactccttcagt

SEQ ID NO: 12 - DNA, 27 bp, Artificial Sequence
  Description of Artificial Sequence Synthetic primer
gcctggatagcggatacactcgtcaca

SEQ ID NO: 13 - DNA, 20 bp, Artificial Sequence
  Description of Artificial Sequence Synthetic primer
gtgtgtgtcaatggctggac

SEQ ID NO: 14 - DNA, 20 bp, Artificial Sequence
  Description of Artificial Sequence Synthetic primer
cgatagagcactcgtccaca

SEQ ID NO: 15 - DNA, 20 bp, Artificial Sequence
  Description of Artificial Sequence Synthetic primer
ggcttctactccgcttcctt

SEQ ID NO: 16 - DNA, 20 bp, Artificial Sequence
  Description of Artificial Sequence Synthetic primer
caacttctgcctttggcttc

SEQ ID NO: 17 - DNA, 20 bp, Artificial Sequence
  Description of Artificial Sequence Synthetic primer
ctaccccagccagtgtcaac

SEQ ID NO: 18 - DNA, 20 bp, Artificial Sequence
  Description of Artificial Sequence Synthetic primer
tcagctggctcagactttca

SEQ ID NO: 19 - DNA, 25 bp, Artificial Sequence
  Description of Artificial Sequence Synthetic primer
cgacaccggataaaccaaagacagc

(19) United States
(12) Patent Application Publication
(10) Pub. No.: US 2006/0194315 A1
(43) Pub. Date: Aug. 31, 2006

(54) Compositions and methods for the control, differentiaton and/or manipulation of pluripotent cells through a gamma-secretase signaling pathway

(76) Inventors: Brian G. Condie, ATHENS, GA (US); Allan J. Robins, Athens, GA (US); Scott Allen Noggle, New York, NY (US)

Correspondence Address: Sutherland, Asbill & Brennan/Atta: Bill Warren, 999 Peachtree Street, NE, Atlanta, GA, 30309-3996, US

(21) Appl. No.: 10/551,876

(22) PCT Filed: Mar. 31, 2004
(86) PCT No.: PCT/US04/09817

§ 371 Date: Jan. 20, 2006

Related U.S. Application Data
(60) Provisional application No. 60/459,129, filed on Mar. 31, 2003.
(60) Provisional application No. 60/516,582, filed on Oct. 31, 2003.

(51) Int. Cl. C12N 5/08

(52) U.S. Cl. 435/366

(57) ABSTRACT

The current invention relates to the control and/or manipulation of the gamma-secretase signaling pathway in pluripotent cells to stabilize the cells in a pluripotent state and/or to control the differentiation of the pluripotent cells towards a differentiated state. The invention further includes feeder layers that contain or express ligands or other compounds that inhibit gamma-secretase or Notch signaling to enhance the maintenance of pluripotent cells in a pluripotent state. The invention also includes cell culture compositions that comprise pluripotent cells and inhibitors of gamma-secretase, or activators or inhibitors of Notch signaling.

### ACKNOWLEDGMENT OF FEDERAL RESEARCH SUPPORT

This invention was made, at least in part, with funding from the National Institutes of Health (Grant Number 2-R24-DK63689-01). Accordingly, the United States Government has certain rights in this invention.

### BACKGROUND OF THE INVENTION

1. Field of the Invention

This invention relates generally to the control, differentiation and/or manipulation of pluripotent cells via modulation of the gamma-secretase or Notch signaling pathways.

2. Background Art

The successful isolation, long term clonal maintenance, genetic manipulation and germ-line transmission of pluripotent cells from species other than rodents has generally been difficult and the reasons for this are unknown. International Patent Application WO 97/32033 and U.S. Pat. No. 5,453,357 describe pluripotent cells including cells from species other than rodents. Human ES cells have been described in International Patent Application WO 96/23362, and in U.S. Pat. No. 5,843,780, and human EG cells have been described in International Patent Application WO 98/43679.

The ability to tightly control differentiation or form homogeneous populations of partially differentiated or terminally differentiated cells by differentiation in vitro of pluripotent cells has proved problematic. Most current approaches involve the formation of embryoid bodies from pluripotent cells in a manner that is not controlled and does not result in homogeneous populations. Mixed cell populations such as those in embryoid bodies of this type are generally unlikely to be suitable for therapeutic or commercial use.

Uncontrolled differentiation produces mixtures of pluripotent stem cells and partially differentiated stem/progenitor cells corresponding to various cell lineages. When these ES-derived cell mixtures are grafted into a recipient tissue the contaminating pluripotent stem cells proliferate and differentiate to form tumors, while the partially undifferentiated stem and progenitor cells can further differentiate to form a mixture of inappropriate and undesired cell types. It is well known from studies in animal models that tumors originating from contaminating pluripotent cells can cause catastrophic tissue damage and death. In addition, pluripotent cells contaminating a cell transplant can generate various inappropriate stem cell, progenitor cell and differentiated cell types in the donor without forming a tumor. These contaminating cell types can lead to the formation of inappropriate tissues within a cell transplant. These outcomes cannot be tolerated for clinical applications in humans. Therefore, uncontrolled ES cell differentiation makes the clinical use of ES-derived cells in human cell therapies impossible.

Hence it is extremely desirable to have an improved method that stabilized pluripotent cells in a pluripotent state and/or an improved method to control the differentiation of pluripotent cells to produce the desired cell type(s).

Alterations in Notch signaling can affect cell proliferation. Mutations or experimental manipulations that alter Notch expression and/or function can lead to or are associated with neoplastic transformation and the generation and uncontrolled proliferation of tumor cells (Jhappen et al., 1992 Genes and Development 6:345-355; Robbins et al., 1992 Journal of Virology 66:2594-2599; Ellisen et al., 1991, Cell 66:649-661; Zagouras et al., 1995 Proc. Natl. Acad. Sci. 92:6414-6418; Capobianco et al., 1997, Molecular and Cellular Biology 17:6265-6273; Weijzen et al., 2002 Nature Med. 8:979-986; Joutel et al., 1998, Seminars in Cell and Developmental Biology 9:619-625; Jang et al., 2000 Current Opinion Molecular Therapy 2:55-65). The role of Notch signaling in tumor formation and cellular transformation indicates that Notch signaling can stimulate cell proliferation. In addition, recent work has shown that Notch can act as a tumor suppressor. In this case, the genetic deletion of Notch specifically in the skin leads to cellular transformation and the production of tumors (Nicolas et al., 2003, Nature Genetics 33:416-421). This result suggests that Notch can also act as a negative regulator of cell proliferation and cellular transformation. A related observation is that the genetic deletion of Notch in keratinocytes leads to increased proliferation (Rangarajan et al, 2001, EMBO Journal 13:3427-3436). These observations indicate that Notch could act to negatively or positively regulate the proliferation of a wide variety of cell types including embryonic stem cells. These changes in the proliferation rate can either be linked to or completely independent of changes in the differentiation state of the embryonic stem cells.

Recently, two articles have compared the transcriptional profiles of mouse embryonic, neural and hematopoeitic cells (Ivanova et al., 2002, Science 298:601-604; Ramalho-Santos et al., 2002, Science 298:597-600). The article compared hundreds of genes expressed by these different cell types. The article noted that the three classes of stem cells were enriched for members of the Notch signaling pathway. However, despite listing hundreds of genes expressed by murine stem cells, the article gives no guidance or insight into genes that may be useful for the maintenance or differentiation of human pluripotent cells.

U.S. Pat. No. 5,780,300 provides a method for the expansion of non-terminally differentiated cells using activators of Notch signaling. However, this document does not teach or suggest that the Notch pathway could be used to control, stabilize or otherwise manipulate pluripotent cells as desired.

PCT Publication No. WO 02/7204 discloses utilizing Notch with embryonal carcinoma (EC) cell lines. While the patent application discloses that the invention can extend to include embryonic stem (ES) cells, the patent application has not demonstrated that the invention does extend to ES cells, and particularly to human ES cells. Because of the numerous fundamental differences between EC and ES cells, it is not predictable that the relationship of Notch in EC cells would translate to ES cells.

Notch signaling can also be modulated by altering the activity of the gamma-secretase complex. This complex is required for the cleavage of the Notch receptor releasing the Notch intracellular domain (reviewed in Fortini, 2002, Nature Reviews Molecular and Cell Biology 3, 673-684). Gamma-secretase inhibitors have been used to reduce the level of Notch signaling and lead to effects that resemble or are identical to the phenotypes produced by loss of function mutations in Notch genes in a variety of organisms and experimental systems (Dovey et al, 2001, Journal of Neurochemistry 76, 173-181; Hadland et al, 2001, Proceedings of the National Academy of Sciences USA 98, 7487-7491; Doerfler et al., 2001, Proceedings of the National Academy of Sciences USA 98, 9312-9317; Micchelli et at, 2002, The FASEB Journal 17:79-81). It is anticipated that additional small molecule modulators of gamma-secretase activity will be developed or discovered including agonists or inducers. In addition the genes encoding the components of the gamma-secretase complex or the proteins themselves including Presenilin, nicastrin, aph-1, pen-2 and related proteins (Fortini, 2002, Nature Reviews Molecular Cell Biology 3, 673-684) could be introduced into cells to modulate the activity of the gamma-secretase complex. Increasing the level of these proteins may up-regulate Notch cleavage while the introduction of mutant genes or proteins could result in constitutively active gamma-secretase activity or in a reduction of gamma-secretase activity. These changes in gamma-secretase activity could alter the level of Notch signaling in the embryonic stem cells.

While, the gamma-secretase complex can signal through the Notch pathway (De Strooper et al., 1999 Nature 398:518-522), it can also signal through a number of other pathways. The number of substrates for gamma-secretase cleavage is growing. Evidence for a signaling pathway based on regulated intra-membrane proteolysis of type I integral membrane proteins that can relay extra-cellular signals by the generation of transcriptionally active intracellular fragments is emerging (reviewed in Medina & Dotti, 2003 Cell Signal, 15(9):829-41). In addition to the well described actions on Notch and Abeta precursor protein, these substrates include Delta and Jagged (Ikeuchi & Sisodia, 2003 J. Biol. Chem. 278:7751-7754; LaVoie & Selkoe, 2003 J. Biol. Chem. 278(36):34427-37), ErbB-4 (Lee et al., 2002 J. Biol. Chem. 277:6318-6323; Ni et al., 2001 Science 294:2179-2181), CD44 (Lammich et al., 2002 J. Biol. Chem. 277:44754-44759), LDL receptor-related protein (May et al., 2002 J. Biol. Chem. 277:18736-18743), E/N-cadherin (Marambaud et al., 2002 EMBO J. 21:1948-1956), Nectin-1 (Kim et al., 2002 J. Biol. Chem. 277:49976-49981), APP (De Strooper et al., 1998 Nature 391:387-390) and APLP1/2 (Scheinfeld et al., J Biol. Chem. 2002; 277:44195-201). Of the possible released fragments only the Notch ICD has been shown to regulate gene expression. In addition, gamma-secretase cleavage may regulate other biological functions. For example, gamma-secretase cleavage of E-cadherin may regulate adherins junction disassembly (Marambaud et al., 2002 EMBO J. 21:1948-1956). This cleavage may also have an indirect effect on gene expression by regulating the transcriptional signaling pool of beta-catenin, an effector of the wingless signaling pathway.

There is a need, therefore, to identify methods and compositions for the more effective control, maintenance and manipulation of pluripotent cells.

### SUMMARY OF THE INVENTION

It is an object of the present invention to overcome, or at least alleviate, one or more of the difficulties or deficiencies associated with the prior art. In that regard, it has been demonstrated that the active form of certain components of the gamma-secretase complex are expressed in human embryonic stem (hES) cells. In addition, it has been observed that the inhibition of the active forms of components of the gamma-secretase complex stabilizes human pluripotent cells in an undifferentiated state, and reduces the percentage of spontaneously differentiated cells in the pluripotent cell culture. This discovery suggests that gamma-secretase is key in at least one signaling pathway used to stabilize human pluripotent cells in a pluripotent state.

In that regard, the present invention provides methods and compositions for controlling or manipulating pluripotent cells as desired via the gamma-secretase and Notch signaling pathway. The invention provides a cell culture composition comprising pluripotent cells and an inhibitor of at least one component of the gamma-secretase complex. In certain embodiments, the inhibitor of at least one component of the gamma-secretase complex is selected from the group consisting of non-transition state analogues, transition state analogs, helical peptides containing α-aminoisobutrric acid, Fenchylamine Sulfonamide compounds, NSAIDs, and benzodiazepines. In one embodiment, the inhibitor comprises DAPT. In another embodiment, the inhibitor comprises a transition state analog selected from the group consisting of III-31-C, L-685,458, and a substrate-based difluroketone peptidomimetic. In a further embodiment, the substrate-based difluroketone peptidomimetic is DFK-167. In one embodiment, the inhibitor of at least one component of the gamma-secretase complex inhibits Notch signaling in the pluripotent cells.

The invention further provides for a cell culture composition comprising pluripotent cells and an inhibitor of Notch signaling. In one embodiment, the inhibitor of Notch signaling is selected from the group consisting of a gamma secretase inhibitor, and a dominant negative Notch protein. In another embodiment, the dominant negative Notch protein comprises an extracellular domain of one or more Notch proteins or a portion thereof.

The invention further provides for a cell culture composition comprising pluripotent cells and an activator of Notch signaling. In one embodiment, the activator is a ligand selected from the group consisting of Jagged-1, Jagged-2, Jagged-3, Serrate, any member of the Jagged/Serrate protein family, Delta, Delta-like-1, Delta-like-3, Delta-like-4, Delta-like homolog-1 (DLK1); any member of the Delta protein family; and any portion of any of these proteins. In another embodiment, a majority of the cells are differentiated after culture with the activator. In one embodiment, the cells are differentiated into neural cells.

The invention further provides for methods of differentiating or stabilizing human pluripotent cells, wherein said methods comprise: (a) providing human pluripotent cells that express one or more Notch proteins, (b) providing an activator or inhibitor of at least one of the one or more Notch proteins on the pluripotent cells; and (c) contacting the human pluripotent cells with the activator or inhibitor to thereby differentiate or stabilize the human pluripotent cells. In one embodiment, the invention provides for a method of stabilizing human embryonic stem cells in a pluripotent state, wherein the cells express one or more Notch proteins, wherein said method comprises providing an inhibitor of Notch signaling to thereby stabilize the cells. In another embodiment, the method of stabilizing human pluripotent cells comprises (a) providing a human feeder layer wherein the feeder layer exqpresses an inhibitor of Notch signaling, wherein the inhibitor of Notch signaling is selected from the group consisting of a gamma-secretase inhibitor, and a dominant negative Notch protein; and (b) contacting the human pluripotent cells with the human feeder layer in a culture medium to thereby stabilize the human pluripotent cells in a pluripotent state. In a further embodiment, the invention provides for a method of controlling the differentiation of human pluripotent cells, comprising (a) providing a human feeder layer wherein the feeder layer expresses an activator of Notch signaling; and (b) contacting the human pluripotent cells with the human feeder layer in a culture medium to thereby differentiate the human pluripotent cells.

The invention also encompasses a method of stabilizing a pluripotent cell culture, comprising: (a) providing a pluripotent cell culture; and (b) contacting the pluripotent cell culture with an inhibitor of at least one component of the gamma-secretase complex to thereby stabilize the pluripotent cell culture.

The invention contemplates that the pluripotent cells are human cells. In one embodiment, the human pluripotent cells are selected from the group consisting of human embryonic stem cells, human inner cell mass (ICM)/epiblast cells, human primitive ectoderm cells, and human primordial germ cells. In a further embodiment, the human cells are human embryonic stem cells.

It is contemplated that when the cells are stabilized, they are stabilized in a pluripotent state for at least 10 passages. It is also contemplated that the pluripotent state can be determined by the expression of markers characteristic for pluripotency, such as by expression of SSEA4 and Notch1. In one embodiment, SSEA4 and Notch 1 are expressed in at least approximately 60% of the cells. In another embodiment, the pluripotent state is assessed by the lack of expression of markers characteristic of differentiated cells, such as by expression of HNF4alpha. In one embodiment, less than approximately 20% of the cells express HNF4alpha after approximately 10 passages.

### BRIEF DESCRIPTION OF THE DRAWINGS

FIGS. 1A-F show Notch1 expression in undifferentiated and differentiating BGN1 hES cells. A-C (400×) show undifferentiated manually passaged human ES colony, Notch 1 is highly expressed on the surface of morphologically undifferentiated hES cells (B). These cells also express SSEA4 (C). D-E (600×) show a differentiating region of a manually passaged human ES colony. Differentiating cells in this area of the colony are negative for Notch1(E, arrowheads) and SSEA4 (F, arrowheads) that are can be seen adjacent to cells that are still positive for Notch1 (E, arrows) and SSEA4 (F, arrows).

FIGS. 2A-D show SSEA4 selection of Trypsin passaged BGN1 hES cells. Morphology of Trypsin passaged cells after two passages (A, 200×). Colonies grew without well defined borders. However, at higher magnification of A (B, 400×), the cells maintain a morphology similar to that of manually passaged hES cells. At passage 8, in addition to the first colony type, a small number of colonies with a compact dome morphology appeared (C, 200×). Magnetic sorting for SSEA4 expression enriched for the compact dome colony morphology (D, 400×). Oct4 staining of a colony from the retained fraction (E,, 400×) and the flow-through (F, 400×) from the SSEA4 magnetic sorting procedure.

FIGS. 3A-L show SSEA4 selected hES cells have a pluripotent antigenic profile. Colonies of BGN1 hES cells magnetically enriched for SSEA4 expression express Oct-4 (D), SSEA3 (F), SSEA4 (J), TRA-1-60 (K), and TRA1-81 (L), but not SSEA1 (E). Images A-C and G-I are DAPI nuclear counterstains of images D-F and J-L respectively. All images are at 200× magnification.

FIGS. 4A-G show SSEA4 selected cells also express Notch1 and rapidly downregulate expression upon differentiation. Undifferentiated SSEA4 selected BGN1 hES cells are uniformly stained with a monoclonal antibody recognizing the intracellular domain of Notch1(bTAN20) (C). As the colonies begin spontaneous differentiation they lose surface staining as shown by staining with a polyclonal antibody that recognizes an extracellular epitope of Notch-1(H-131) (D) Cells at the edge of the colony (bottom of D) lose surface expression of Notch 1. Images A and B are DAPI nuclear counterstains of images C and D. Differentiating cells in colonies of selected sells lose SSEA4 (G) expression as well as Notch-1 (F) (E is a DAPI counterstain of F). All images are at 600× magnification.

FIGS. 5A-B show Deltex RT-PCR. (A) A band of the expected size (267 bp) is found in SSEA4 selected BGN1 hES cells and not in human fibroblast and stromal cell lines. (Lanes 1, 10) Marker, (Lane 2) No template control, (Lane 3) SSEA4 selected BGN1 hES cells, (Lane 4) BJ fibroblasts, (Lane 5) HS27, (Lane 6) HUVEC, (Lane 7) JEG, (Lane 8) KEL fibroblasts, (Lane 9) WS 1. Control reactions that omitted the RT were all negative. (B) Manually passaged BGN1 hES cells also express Deltex (Lane 1), (Lanes 1,6) Marker, (Lane 2) No template control, (Lane3) No RT control for SSEA4 selected BGN1 hES cells (Lane 4). The larger band in lane 4 is a nonspecific product and is not found in the No RT control.

FIGS. 6A and 6B show SSEA4 and Notch1 stains of manually passaged hES cells showing morphologically undifferentiated (below dashed line) and differentiated areas (above dashed line). FIGS. 6C-E show DAPI, SSEA4 and Notch1 stains of trypsin passaged hES cells showing a similar progression as shown by the manually passaged cells. FIG. 6F shows flow analysis for SSEA4/Notch1 showing equivalent populations. Fr. A indicates the SSEA4high/Notch1high fraction, or undifferentiated cells; Fr. B indicates the SSEA4low-neg/Notch1pos fraction, or differentiating cells; Fr. C indicates the SSEA4low-neg/Notch1neg fraction, or differentiated cells. These figures show progression of marker expression upon differentiation and point out that both culture systems produce cell populations that are heterogenous.

FIGS. 7A-H show that hES cells express Notch-1, -2, and -3 and active forms of components of the gamma-secretase complex and can be activated to cleave a substrate upon EDTA exposure. FIG. 7A provides RT-PCR data showing expression of Notch-1, -2, and -3 and weak expression of Notch-4 in hES cells. FIG. 7B shows a Western blot for Notch-1 showing expression in BEGN1 hES cells but not in the MEF feeders. FIGS. 7C-F, respectively, show Western blots indicating that Notch-2, E-cadherin, the mature form of Nicastrin, and the processed CTF form of Presinilin-1 are expressed in hES cells. FIGS. 7G and 7H show Western blots showing that gamma-secretase complex is functional. 7G shows Western blots for Notch1 (using the bTAN20 antibody) and for the gamma-secretase cleaved form of Notch (NICD) under Trypsin/EDTA exposure (lanes 1 and 2 are duplicates) and non-EDTA-collagenase exposure (lane 3). Oct4 expression demonstrates a pluripotency marker and HDAC expression was used as a loading control. FIG. 7H shows that Notch cleavage can be inhibited by treatment with a gamma-secretase inhibitor, DAPT. DMSO was shown as a control. The cleaved form of Notch is not generated with exposure to Trypsin/EDTA in the presence of DAPT, but is generated in the presence of DMSO, as evidenced by absence of the 110 kd band in the Notch1 blot using the bTAN20 antibody and the NICD blot. The asterisk shows a non-specific band found only in the MEF feeders. HDAC is shown as loading control.

FIG. 8 shows that activation of Notch signaling can upregulate expression of the Notch target gene Hes1 in hES cells. This activation can be repressed by inhibitors of gamma-secretase. FIG. 8 shows graph of real-time RT-PCR data for Hes1 activation and gamma-secretase inhibitor repression of Hes1 activation. GAPDH normalized Hes1 expression ratio with EDTA induction in the absence (DMSO) or presence (DAPT) of the gamma-secretase inhibitor are compared. The values were log transformed for statistical analysis. The reduction in Hes1 expression with inhibitor treatment is significant (p=0.032, t-test).

FIG. 9 shows the experimental design of gamma-secretase inhibitor treatment experiments.

FIGS. 10A-J show the flow analysis of DAPT treated vs. DMSO treated cultures examining SSEA4 and Notch1 expression. FIG. 10A shows the SSEA4 selected parent culture of hES cells. This culture was further passaged, and SSEA4 and Notch1 were analyzed by flow cytometry for untreated cells (FIGS. 10B-D), DMSO treated cells (FIGS. 10E-G), and DAPT treated cells (FIGS. 10H-J). The groups were set up in triplicate. DAPT treatment reduced the proportion of Fr. B (SSEA4low-neg/Notch1pos) cells in culture. Numbers shown are the percentage in the region. The samples were gated to exclude debris, but included all cells based on side scatter/forward scatter characteristics.

FIGS. 11A-D show a summary of SSEA4/Notch1 flow cytometry data for the experiment shown in FIG. 5. Data are from 4 experiments across two human ES cell lines, BGN1 and BGN2. Both the number (11A) and proportion (11B) of cells in Fr. B are significantly reduced with inhibitor treatment. The proportion of cells in Fr. A (11D) is significantly increased with inhibitor treatment while the number of cells in this fraction remained unchanged (11C). The number of cells in each fraction was obtained by multiplying the percent of cells in the fraction by the total yield of cells harvested from the dish.

FIGS. 12A-H show that inhibition of gamma-secretase dependent signaling stabilizes hES cells in an undifferentiated state under these passaging conditions, and reduces the number of spontaneously differentiated cells in the culture. 12A and 12B show SSEA4 immunohistochemical analysis of DMSO and DAPT treated cultures. 12C and 12D show DAPI stains of the same cultures. FIGS. 12E-H show the morphology of EBs generated from late trypsin passaged cultures maintained in DAPT (12G) vs. DMSO (12E) vs. untreated derived EBs (12F) compared to manually passaged derived EBs (12H). Note that manually passaged derived EBs are cystic, whereas the untreated trypsin passaged derived EBs are not cystic. DAPT treatment of trypsin passaged cultures returns the EBs to a cystic morphology resembling the manual passaged derived EBs.

### DETAILED DESCRIPTION OF THE INVENTION

Applicant has demonstrated that contacting pluripotent human cells such as human ES cells, with at least one compound that inhibits at least one component of the gamma-secretase complex improves the ability of the cells to be stabilized in a pluripotent state and reduces the spontaneous differentiation of the cell culture. In that regard, the invention provides a cell culture composition comprising pluripotent cells and an inhibitor of at least one component of the gamma-secretase complex.

The invention further provides for a cell culture composition comprising pluripotent cells and an inhibitor of Notch signaling. In one embodiment, the inhibitor of Notch signaling is selected from the group consisting of a gamma-secretase inhibitor, and a dominant negative Notch protein. In another embodiment, the dominant negative Notch protein comprises an extracellular domain of one or more Notch proteins or a portion thereof.

The invention further provides for a cell culture composition comprising pluripotent cells and an activator of Notch signaling. In one embodiment, the activator is a ligand selected from the group consisting of Jagged-1, Jagged-2, Jagged-3, Serrate, any member of the Jagged/Serrate protein family, Delta, Delta-like-1, Delta-like-3, Delta-like-4, Delta-like homolog-1 (DLK1); any member of the Delta protein family; and any portion of any of these proteins. In another embodiment, a majority of the cells are differentiated after culture with the activator. In one embodiment, the cells are differentiated into neural cells.

The invention further provides for methods of differentiating or stabilizing human pluripotent cells, wherein said methods comprise: (a) providing human pluripotent cells that express one or more Notch proteins, (b) providing an activator or inhibitor of at least one of the one or more Notch proteins on the pluripotent cells; and (c) contacting the human pluripotent cells with the activator or inhibitor to thereby differentiate or stabilize the human pluripotent cells. In one embodiment, the invention provides for a method of stabilizing human embryonic stem cells in a pluripotent state, wherein the cells express one or more Notch proteins, wherein said method comprises providing an inhibitor of Notch signaling to thereby stabilize the cells. In another embodiment, the method of stabilizing human pluripotent cells comprises (a) providing a human feeder layer wherein the feeder layer expresses an inhibitor of Notch signaling, wherein the inhibitor of Notch signaling is selected from the group consisting of a gamma-secretase inhibitor, and a dominant negative Notch protein; and (b) contacting the human pluripotent cells with the human feeder layer in a culture medium to thereby stabilize the human pluripotent cells in a pluripotent state. In a further embodiment, the invention provides for a method of controlling the differentiation of human pluripotent cells, comprising (a) providing a human feeder layer wherein the feeder layer expresses an activator of Notch signaling; and (b) contacting the human pluripotent cells with the human feeder layer in a culture medium to thereby differentiate the human pluripotent cells.

The present invention encompasses a method of controlling, stabilizing or manipulating human pluripotent cells, wherein said method comprises providing human pluripotent cells expressing at least one active form of a component of the gamma-secretase complex, and providing a compound or altering the surroundings of said pluripotent cells to activate or deactivate said gamma-secretase complex on said pluripotent cells. In one embodiment, the invention encompasses a method of stabilizing a pluripotent cell culture, comprising: (a) providing a pluripotent cell culture; and (b) contacting the pluripotent cell culture with an inhibitor of at least one component of the gamma-secretase complex to thereby stabilize the pluripotent cell culture.

The invention contemplates that the feeder cell layer can express the inhibitor of at least one component of the gamma-secretase complex, the inhibitor of Notch signaling, or the activator of Notch signaling. The feeder cell layer can be genetically engineered to express any of these inhibitors or activators. In one embodiment, expression of the inhibitor or activator is induced upon the addition of a compound to culture medium for the feeder cell layer.

The present invention contemplates that the inhibitor of at least one component of the gamma-secretase complex is selected from the group consisting of non-transition state analogues such as DAPT or compound E, transition state analogues, helical peptides containing α-aminoisobutyric acid (Aib), Fenchylamine Sulfonamide compounds, NSAIDs, and benzodiazepines. In one embodiment, the inhibitor comprises DAPT, or N-[N-(3,5-Difluorophenacetyl-L-alanyl)]-S-phenylglycine t-Butyl Ester as described in Dovey et al., 2001 J. Neurochem 76:173-181. In another embodiment, the inhibitor comprises compound E as described in Seiffert et al., 2000 JBC 275:34086-91. Preferably the transition state analog is selected from the group comprising III-31-C (Esler et al., 2002 PNAS 99:2720-2725), L-685,458 (Shearman et al., 2000 Biochemistry 39:8698-8704) and a substrate-based difluroketone peptidomimetic. In one embodiment, the substrate-based difluroketone peptidomimetic is DFK-167 or a compound similar to DFK-167 (See, Wolfe et al., 1999 Biochemistry 38:4720-4727; Esler et al., 2000 Nat. Cell Biol. 2:428-434). In another embodiment, the inhibitor comprises a helical peptide containing α-aminoisobutyric acid (Aib), designed to mimic transmembrane regions of substrates (Das et al., 2003 J Am. Chem. Soc. 125:11794-11795). In yet another embodiment, the inhibitor comprises a Fenchylamine Sulfonamide compound such as, but not limited to, those compounds described in Rishton et al., 2000 J Med. Chem. 43:2297-2299. In another embodiment, the inhibitor comprises an NSAID, such as, but not limited to ibuprofen, flurbiprofen, and its enantiomers. In another embodiment, the inhibitor comprises a benzodiazepine, such as, but not limited to those described in Churcher et al., 2003 Bioorg. Med. Chem. Lett. 13:179-183 and Churcher et al., 2003 J Med. Chem. 46:2275-78. In one embodiment, the inhibitor of at least one component of the gamma-secretase complex inhibits Notch signaling in the pluripotent cells.

It is contemplated that the pluripotent state of the cells can be determined examination of the cell morphology and expression patterns of the cells. In one embodiment, treatment of the pluripotent cells with the inhibitor of at least one component of the gamma-secretase complex or with the inhibitor of the Notch signaling pathway stabilizes the cells in a pluripotent state. Pluripotency can be determined, for example, by examining expression of the markers SSEA4 and Notch1. In one embodiment, at least approximately 50% of the pluripotent cells treated with the inhibitor express SSEA4 and Notch1. More preferably, at least approximately 55%, more preferably, at least approximately 60%, 70%, 75%, 80%, 85%, 90%, or at least approximately 95% of the cells express SSEA4 and Notch1. Alternatively, pluripotency may be determined by examining differentiation markers. Differentiation markers are specific for a differentiation pathway. For example, differentiation along the endodermal pathway may be determined by examining, for example, expression of HNF4alpha or GATA-4 in the cell culture. In one embodiment, less than approximately 30% of the pluripotent cells treated with the inhibitor express an endodermal differentiation marker. More preferably, less than approximately 25%, less than 20%, less than 15%, less than 10% or less than 5% of the cells treated with the inhibitor express an endodermal differentiation marker.

It is contemplated that the inhibitor may be expressed from a feeder cell layer. In one embodiment, the feeder cell layer is genetically engineered to express the inhibitor of gamma-secretase or Notch signaling, or the activator of Notch signaling. In another embodiment, the inhibitor of gamma-secretase or Notch signaling, or the activator of Notch signaling is added to the culture medium. In another embodiment, expression of the inhibitor of gamma-secretase or Notch signaling, or the activator of Notch signaling is induced by the addition of a compound to the culture medium for the feeder cell layer.

In another embodiment, the invention provides for a method of controlling or manipulating the differentiation of human pluripotent cells, comprising adding a modulator of at least one component of the ganuna-secretase complex or the Notch signaling pathway to allow the pluripotent cells to go from a pluripotent state towards a more differentiated state. In certain embodiments of the above-described methods, the pluripotent cells are directed down a neural cell pathway.

Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5th ed, Berlin: Springer-Verlag; in Current Protocols in Molecular Biology, F. M. Ausubel et al., Eds., Current Protocols, a joint venture between Greene Publishing Associates, Inc. and John Wiley & Sons, Inc., (1998 Supplement); in Current Protocols in Cell Biology, J. S. Bonifacino et al., Eds., Current Protocols, John Wiley & Sons, Inc. (1999 Supplement); and in Current Protocols in Neuroscience, J. Crawley et al., Eds., Current Protocols, John Wiley & Sons, Inc. (1999 Supplement). It is to be understood that as used in the specification and in the claims, “a” or “an” can mean one or more, depending upon the context in which it is used. Thus, for example, reference to “a cell” can mean that at least one cell can be utilized and reference to “an inhibitor” means that at least one inhibitor can be utilized.

As used herein when referring to a cell, cell line, cell culture or population of cells, the term “isolated” refers being substantially separated from the natural source of the cells such that the cell, cell line, cell culture, or population of cells are capable of being cultured in vitro.

As used herein, the term “express” refers to the transcription of a polynucleotide or translation of a polypeptide in a cell, such that levels of the molecule are measurably higher in a cell that expresses the molecule than they are in a cell that does not express the molecule. Methods to measure the expression of a molecule are well known to those of ordinary skill in the art, and include without limitation, Northern blotting, RT-PCT, in situ hybridization, Western blotting, and immunohistochemistry.

Preferably, the pluripotent cells are selected from the group consisting of embryonic stem cells, ICM/epiblast cells, primitive ectoderm cells, primordial germ cells, and teratocarcinoma cells. In a preferred embodiment, the pluripotent cell is a human cell. As used herein, the term “pluripotent human cell” encompasses pluripotent cells obtained from human embryos, fetuses or adult tissues. In one preferred embodiment, the pluripotent human cell is a human pluripotent embryonic stem cell. In another embodiment the pluripotent human cell is a human pluripotent fetal stem cell, such as a primordial germ cell or EG cell. In another embodiment the pluripotent human cell is a human pluripotent adult stem cell. As used herein, the term “pluripotent” refers to a cell capable of at least developing into one of ectodermal, endodermal and mesodermal cells. As used herein the term “pluripotent” refers to cells that are totipotent and multipotent. As used herein, the term “totipotent cell” refers to a cell capable of developing into all lineages of cells. The term “multipotent” refers to an undifferentiated cell that can form more than one differentiated cell type. The term “progenitor” refers to an undifferentiated cell that may be multipotent or committed to form a single differentiated cell type.

The human pluripotent cells of the present invention can be derived using any method known to those of skill in the art. For example, the human pluripotent cells can be produced using de-differentiation and nuclear transfer methods. Additionally, the human ICM/epiblast cell or the primitive ectoderm cell used in the present invention can be derived in vivo or in vitro. EPL cells may be generated in adherent culture or as cell aggregates in suspension culture, as described in WO 99/53021, herein incorporated by reference in its entirety. Furthermore, the pluripotent cells can be passaged using any method known of those to skill in the art, including, manual passaging methods, and bulk passaging methods such as antibody selection and protease passaging.

As used herein, the term “contacting” (i.e., contacting a cell e.g. a pluripotent cell, with an compound) is intended to include incubating the compound and the cell together in vitro (e.g., adding the compound to cells in culture). The term “contacting” is not intended to include exposure of pluripotent cells to a modulator of the gamma-secretase or Notch signaling pathways that may occur naturally in a subject (i.e., exposure that may occur as a result of a natural physiological process). The step of contacting the pluripotent cell or cell culture with the compound to modulate the gamma-secretase or Notch signaling pathway can be conducted in any suitable manner. For example, the pluripotent cells may be treated in adherent culture, or in suspension culture. The pluripotent cells may have been formed into embryoid bodies prior to or during exposure to the compound. Embryoid bodies may be generated in suspension culture using the hanging drop technique or by culturing the cells on agarose coated plates. It is understood that the cells treated with the compound to modulate the gamma-secretase or Notch signaling pathway may be further treated with other cell differentiation environments to stabilize the cells in a pluripotent state, or to differentiate the cells further. In one embodiment, the cells treated with the compound are further differentiated into neural cells.

As used herein, the term “neural cell” includes, but is not limited to, neurectoderm cells; EPL-derived cells, glial cells; neural cells of the central nervous system such as a dopaminergic cell, differentiated or undifferentiated astrocytes or an oligodendrocytes, neural stem cells, neuronal progenitors, glial progenitors, differentiated neurons such as dopaminergic neurons, and a neural cell of the peripheral nervous system. As used herein, the term “neurectoderm” refers to undifferentiated neural progenitor cells substantially equivalent to cell populations comprising the neural plate and/or neural tube. Neurectoderm cells are multipotential. The neural cell types that differentiate from embryonic stem cells have several uses in various fields of research and development including but not limited to drug discovery, drug development and testing, toxicology as well as basic science research. These useful neural cell types include neurons of a wide variety of morphologies and phenotypes as well as various types of glial cells such as astrocytes and oligodendrocytes. These cell types express molecules that are of interest in a wide range of research fields. These include the molecules known to be required for the functioning of neural cell types as described in standard reference texts and current reviews on neurobiology and neurophysiology (Cooper, Bloom et al., 1996; Fain 1999; Kandel, Schwartz et al., 2000; Khakh 2001; Bowery, Bettler et al., 2002; Howlett, Barth et al., 2002; Laube, Maksay et al., 2002). These molecules include but are not limited to cytokines, growth factors, neurotrophic factors, neuroactive peptides (neuropeptides), cytokine receptors, growth factor receptors, ionotropic and metabotropic neurotransmitter receptors, neurotransmitter transporters including plasma membrane reuptake transporters as well as vesicular neurotransmitter transporters, voltage gated ion channels, and ion pumps. The neural cells also express the enzymes in the biochemical pathways that produce and degrade various neurotransmitters.

In a one embodiment the cells that are maintained or stabilized are early primitive ectoderm like or EPL cells as described in WO 99/53021. As used herein, the term “stabilize” refers to the differentiation state of a cell or cell line. When a cell or cell line is stabilized in culture, it will continue to proliferate over multiple passages in culture, and preferably indefinitely in culture; additionally, each cell in the culture is preferably of the same differentiation state, and when the cells divide, typically yield cells of the same cell type or yield cells of the same differentiation state. Preferably, a stabilized cell or cell line does not further differentiate or de-differentiate if the cell culture conditions are not altered, and the cells continue to be passaged and are not overgrown. In one embodiment of the present invention, the compound stabilizes the cell in culture for more than 2 passages, preferably for more than about 5 passages, more preferably for more than about 10 passages, and most preferably for more than about 20 passages.

As used herein, the term “modulate” refers to the ability to stimulate, increase or upregulate a particular response or activity and/or the ability to inhibit, decrease, or downregulate a particular response or activity. Inhibitors and activators of the gamma-secretase signaling pathway are both modulators of gamma-secretase signaling. Similarly, inhibitors and activators of the Notch signaling pathway are both modulators of Notch signaling. It is recognized that modulators of gamma-secretase signaling can also be modulators of Notch signaling.

An activator of the gamma-secretase complex is an agent that promotes activation of gamma-secretase signaling through any of its possible signaling pathways. An inhibitor of the gamma-secretase complex is an agent that antagonizes gamma-secretase signaling through any of its possible signaling pathways. For example, gamma-secretase may signal through the Notch pathway, through the Abeta precursor protein, Delta and Jagged, ErbB4, CD44, LDL receptor-related protein, E/N-cadherin, Nectin-1, APP, and APLP1/2, may signal through the wingless/wnt signaling pathway, as well as regulating adherins junction disassembly.

The compound used to inhibit an active form of one or more components of the gamma-secretase complex may be any compound known in the art, or later discovered. As used herein, the term “inhibitor of gamma-secretase signaling” means that the compound decreases signaling of at least one component of the gamma-secretase complex, known now or later discovered.

Genetic and molecular studies have led to the identification of a group of genes that define distinct components of the gamma-secretase signaling pathway. It is contemplated that the pluripotent cells express at least one of the components of the gamma-secretase signaling pathway.

Activators of the gamma-secretase pathway are able to stimulate the gamma-secretase pathway at the level of protein-protein interaction or protein-DNA interaction. Activators of gamma-secretase include, but are not limited to, the “activated” forms of the Notch receptor. This “activated” Notch receptor corresponds to the metalloprotease-cleaved membrane associated Notch. Its also sometimes called Notch-DE (See Schroeter et al., 1998 Nature 393:382-386). Other substrates may have analogous “activated” forms, as many of them are cleaved by metalloproteases. Such other substrates may include TACE, BACE, ADAM10, or Kuzbanian.

Gamma-secretase inhibition or activation is preferably carried out by contacting an embryonic stem cell with a modulator of gamma-secretase. Preferably, the modulator of gamma-secretase inhibits the signaling of the gamma-secretase complex. The inhibitor can be a soluble molecule, recombinantly expressed as a cell-surface molecule, expressed on a feeder cell layer with which the embryonic stem cells are contacted or a molecule inmnobilized on a solid phase. In another embodiment, the inhibitor can be recombinantly expressed from a nucleic acid introduced into the embryonic stem cells. The inhibitor may be small molecule such as a protein, an antibody, or may be antisense RNA, antisense oligonucleotides, antisense morpholino modified oligonucleotides or RNAi methodologies. These proteins, fragments and derivatives thereof can be recombinantly expressed and isolated, expressed within the cell, expressed on the cell surface of a cell that is placed in contact with the embryonic stem cells or can be chemically synthesized.

An activator of Notch signaling is an agent that promotes activation of one or more Notch proteins or any of their upstream or downstream signaling components through any of its possible signaling pathways. An inhibitor of Notch signaling is an agent that antagonizes the activity of one or more Notch proteins or any of their upstream or downstream signaling components through any of its possible signaling pathways.

The compound used to inhibit or activate Notch signaling can be any compound known in the art, or later discovered. Genetic and molecular studies have led to the identification of a group of genes which define distinct elements of the Notch signaling pathway. While the identification of these various elements has come initially from genetic studies in Drosophila, they have subsequently been found to be active in mammalian systems in a wide variety of developmental contexts (reviewed in Artavanis-Tsakonas et al., 1995, Science 268:225-232; Gridley 1997, Molecular and Cellular Neuroscience 9:103-108; Mumm & Kopan, 2000, Developmental Biology 228:151-165; Kopan, 2002, Journal of Cell Science 115:1095-1097). Genetic studies have identified two Notch pathways that act generally to inhibit gene expression changes (reviewed in Arias et al., 2002, Current Opinion in Genetics and Development 12:524-533). One of these pathways commonly known as the canonical lateral inhibition pathway limits the number of cells that can maintain a set gene expression pattern. The main members involved in this pathway are DSL ligands (members of the Delta, Serrate and Jagged protein families in Drosophila and vertebrates, and Lag-2 in C. elegans), Notch receptors (of which there are four known homologues in mammals), and the CSL transcription factor family members (including Suppressor of Hairless in Drosophila, CBF1 in mammals, and Lag-1 in C. elegans). Current data suggest that when a ligand from the Delta family binds the Notch receptor, the Notch receptor is cleaved on the cytoplasmic side of the membrane releasing an active fragment, the Notch intracellular domain or NICD (Mumm et al., 2000, Mol Cell 5:197-206). NICD translocates to the nucleus where it converts a CSL repressor complex into a CSL activator complex. This new complex acts as a transcriptional activator (reviewed by Mumm & Kopan R, 2000, Dev. Biol. 228:151-165). The canonical target genes include the bHLH transcription factor family of the Enhancer of Split complex (corresponding to the HES gene family in mammals). In Drosophila sensory organ precursor (SOP) development, these transcription factors in turn repress the expression of proneural bHLH transcription factors such as Achaete-Scute. Thus, neural fate is inhibited in cells receiving the Notch signal and the cell expressing the DSL ligand is singled out to become the sensory organ precursor cell.

Activators of the Notch pathway are able to stimulate the Notch pathway at the level of protein-protein interaction or protein-DNA interaction. Activators of Notch include, but are not limited to, proteins and derivatives comprising the portions of the proteins belonging to the Delta or Serrate or Jagged protein families (Lindsell et al., 1995, Cell 80:909-917) that mediate binding to Notch, and nucleic acids encoding the foregoing (which can be administered to express their encoded products in vivo). In a preferred embodiment, the activator is a protein or derivative or fragment thereof comprising a functionally active fragment such as a fragment of a Notch ligand that mediates binding to a Notch protein. In another preferred embodiment, the activator is a human protein or portion thereof (e.g., human Delta). In another preferred embodiment the activator is Deltex or proteins related to Deltex or members of the CSL protein family or a nucleic acid encoding the foregoing (which can be administered to express its encoded product in vivo).

The Notch pathway is a signal transducing pathway comprising elements which interact, genetically and/or molecularly, with the Notch receptor protein. For example, elements which interact with the Notch protein on both a molecular and genetic basis include, but are not limited to, Delta, Serrate, Jagged, Deltex and related proteins. Elements which interact with the Notch protein genetically include, but are not limited to, Mastermind, Hairless, Suppressor of Hairless and CSL transcription factors.

An activator of Notch function is an agent that promotes activation of Notch function. As used herein, “Notch function” shall mean a function mediated by the Notch signaling pathway.

Notch function activation is preferably carried out by contacting an embryonic stem cell with a Notch function activator. The activator of Notch function can be a soluble molecule, recombinantly expressed as a cell-surface molecule, expressed on a cell monolayer with which the embryonic stem cells are contacted or a molecule immobilized on a solid phase. In another embodiment, the Notch activator can be recombinantly expressed from a nucleic acid introduced into the embryonic stem cells. Notch function activator of the present invention include Notch proteins and analogs and derivatives (including fragments) thereof; proteins that are other elements of the Notch pathway and analogs and derivatives (including fragments) thereof; antibodies thereto and fragments or other derivatives of such antibodies containing the binding region thereof; nucleic acids encoding the proteins and derivatives or analogs; as well as proteins and derivatives and analogs thereof which bind to or otherwise interact with Notch proteins or other proteins in the Notch pathway such that Notch function is promoted. Such activator include but are not limited to Notch proteins and derivatives thereof comprising the intracellular domain, Notch nucleic acids encoding the foregoing, and proteins comprising protein domains that interact with Notch (e.g., the extracellular domain of Delta, Serrate or Jagged protein family members). Other activator include Deltex and Suppressor of Hairless and other CSL transcription factors. These proteins, fragments and derivatives thereof can be recombinantly expressed and isolated, expressed within the cell, expressed on the cell surface of a cell that is placed in contact with the embryonic stem cells or can be chemically synthesized.

In a preferred embodiment, the activator is a protein comprising at least a fragment (termed herein “adhesive fragment”) of the proteins which mediate binding to Notch proteins or adhesive fragments thereof. These activator include Notch, Delta, Serrate, Jagged, Suppressor of Hairless/CSL protein family members and Deltex, as well as other members of the Delta/Serrate/Jagged family or Deltex family which may be identified by virtue of sequence homology or genetic interaction and more generally, members of the “Notch cascade” or the “Notch group” of genes and proteins, which are identified by molecular interactions (e.g., binding ini vitro, or biochemical or genetic interactions as demonstrated by, but not limited to, protein-protein interaction in two hybrid or immunoprecipitation assays, genetic studies in organisms such as the mouse, Drosophila, and C. elegans, or assays of Notch functions and interactions in cell cultures).

Vertebrate homologs of the Drosophila Notch pathway elements have been cloned and sequenced. For example, these include Serrate (Lindsell et al., 1995, Cell 80:909-917); Delta (Chitnis et al., 1995, Nature 375:761; Henrique et al., 1995, Nature 375:787-790; Bettenhausen et al., 1995, Development 121:2407); and Notch (Coffinan et al., 1990, Science 249:1438-1441; Bierkamp et al., 1993, Mech. Dev. 43:87-100; Stifani et al., 1992, Nature Genet. 2:119-127; Lardelli et al., 1993, Exp. Cell Res. 204:364-372; Lardelli et al., 1994, Mech. Dev. 46:123-136; Larsson et al., 1994, Genomics 24:253-258; Ellisen et al., 1991, Cell 66:649-661; Weinmaster et al., 1991, Development 113:199-205; Reaume et al., 1992, Dev. Biol. 154:377-387; Weinmaster et al., 1992, Development 116:931-941; Franco del Amo et al., 1993, Genomics 15:259-264; and Kopan et al., 1993, J. Cell. Biol. 121:631-641).

In one embodiment, the Notch activator is expressed from a recombinant nucleic acid. For example, in vivo expression of truncated, “activated” forms of the Notch receptor lacking the extracellular, ligand binding domain result in “gain of function” mutant phenotypes. This is due to the constitutive ligand independent activity of the truncated Notch protein. It has also been mentioned that this process may be desired to be reversible, since when the activated Notch receptor is no longer expressed the embryonic stem cells can respond to differentiation signals and differentiate.

In another embodiment, the recombinantly expressed Notch activator is a chimeric Notch protein comprising the intracellular domain of Notch and the extracellular domain of another ligand-binding surface receptor. For example, a chimeric Notch protein comprising the EGF receptor extracellular domain and the Notch intracellular domain is expressed in a precursor cell. However, the Notch pathway will not be active unless the EGF receptor ligand EGF is contacted with the precursor cell expressing the chimera. As with the inducible promoter controlling the expression of the truncated form of Notch, the activity of the chimeric Notch protein is reversible; when EGF is removed from the cells, Notch activity will cease and the cell can then differentiate. Notch activity can again be turned on with the addition of the ligand.

A systematic deletion analysis of the intracellular domain of Notch demonstrates that the Notch sequences that are both necessary and sufficient for the downstream signaling of the Notch receptor are confined to the ankyrin repeats of the intracellular region (Matsuno et al., 1995, Development 121:2633-2644 and unpublished results). Using the yeast two hybrid system it was discovered that the ankyrin repeats interact homotypically.

Expression of appropriate deletion constructs in the defined cellular environment of the developing Drosophila eye demonstrates that expression of a polypeptide fragment comprising just the ankyrin repeats resulted in an activated phenotype. Not surprisingly this is the part of the Notch protein which is most highly conserved among various species.

These findings suggest that any small molecules, for example, but not by way of limitation, polypeptides or antibodies which bind to the Notch ankyrin repeats, can block its function, and hence behave as inactivators or inhibitors of the pathway. Conversely, molecules that mimic the Notch anlyrin repeat activity can behave as activators of the Notch pathway. Since the expression of truncated forms of Notch give mutant phenotypes in the developing Drosophila eye, genetic screens for modifiers of these phenotypes can be used for identifying and isolating additional gene products that can act as activators or inhibitors of the pathway.

Genes that act as genetic enhancers of the activated phenotypes are potential activators and those that act as genetic suppressors are potential inhibitors.

Deltex and Suppressor of Hairless/CSL protein family members are also activators of Notch function that can be used in the methods and compositions of the present invention. It has been shown that the activation of the Notch pathway, as judged by the induction of activated phenotypes similar to those induced by the expression of activated forms of Notch, can be achieved by manipulating the expression of Deltex (Schweisguth and Posakony, 1994, Development 120:1477), or the expression of Suppressor of Hairless/CSL protein family members (Matsuno et al., 1995, Development 121:2633), both of which can interact with the ankyrin repeats of Notch.

It has recently been shown that Notch signals may function earlier to inhibit the proneural bHLH gene expression pattern independent of CSL mediated signaling. A new class of Notch alleles (Mcd) in Drosophila can result in a gain of function phenotype that is independent of lateral inhibition (Ramain et al., 2001, Curr Biol. 11:1729-1738). In these mutants, bHLH gene expression is never initiated. Deltex, previously thought to be involved in the CSL-dependent Notch signaling, is required for signaling via this non-canonical pathway in Drosophila as lateral signaling occurs normally in flies double mutant for Deltex and the Mcd Notch alleles. Further, the Mcd phenotype cannot be suppressed by loss of Suppressor of Hairless function.

Deltex function can mediate some of the CSL-independent effects of the Notch pathway in vertebrates as well as Drosophila. Overexpression of activated Notch and Deltex in mouse cells and rat neural progenitor cell lines can inhibit the function of the bHLH transcription factors E47 and MASH1, respectively (Ordentlich et al., 1998, Mol. Cell Biol. 18:2230-2239, Yamamoto et al., 2001, J. Biol. Chem. 276:45031-45040). The inhibition of E47 proceeds via inhibition of Ras that normally activates JNK. Consistent with a CSL-independent role, JNK activity is high in Drosophila embryos lacking Suppressor of Hairless-independent Notch function (Zecchini et al., 1999, Curr. Biol. 9:460-469).

Using the yeast ‘interaction trap’ assay (Zervos et al., 1993, Cell 72:223-232), as well as cell culture co-localization studies, the protein regions responsible for heterotypic interactions between Deltex and the intracellular domain of Notch, as well as homotypic interaction among Deltex molecules were defined. The function of the Deltex-Notch interaction domains was examined by ini vivo expression studies. Taken together, data from over-expression of Deltex fragments and from studies of physical interactions between Deltex and Notch demonstrate that Deltex positively regulates the Notch pathway through interactions with the Notch ankyrin repeats.

The Mcd mutations that affect the CSL-independent Notch signaling pathway implicate important regions C-terminal to the intracellular ankyrin repeats and a region within the extracellular EGF-like repeat 18 (Ramain et al., 2001, Curr Biol. 11:1729-1738). These domains are different from those required for the CSL-dependent pathway. The C-terminal region containing the PEST sequence is known to bind Dishevelled, a regulator of Wnt signaling (Axelrod et al., 1996, Science 271:1826-1832, Ramain et al., 2001, Curr Biol. 11:1729-1738). Consequently, Dishevelled is unable to rescue the Mcd mutations as these Notch proteins lack the Dishevelled binding domain. Sites in the N-terminal region of Dishevelled are important for this binding (Axelrod et al., 1996, Science 271:1826-1832). Dishevelled also contains binding sites for Suppressor of Deltex, a ubiquitin ligase, and has been postulated to play a role in regulating Deltex and possibly NICD degradation (Ramain et al., 2001, Curr Biol. 11:1729-1738). Thus, Wnt signaling may antagonize a Deltex mediated Notch signal. Notch intracellular domains that cannot bind Dishevelled may also stabilize the Deltex-Notch complex resulting in maintenance of the undifferentiated state.

In addition, it has been shown that Deltex has a RING-H2 finger domain that mediates homo-oligomerization of Deltex (Matsuno et al., 2002, Development 129:1049-1059). It is possible that Deltex may positively regulate CSL-independent signaling by oligomerizing the Notch receptor. Further, in the Drosophila eye and wing, where antagonism of Wnt and Notch pathways is observed (Strutt et al., 2002 Current Biology 12:813-824, Klein and Martinez Arias, 1998, Developmental Biology 194:196-212), addition of secreted forms of Delta and Serrate block endogenous Notch activity (Sun & Artavanis-Tsakonas, 1997, Development 124:3439-3448, Hukriede et al., 1997, Development 124:3427-3437). It has recently been shown that a soluble Delta ligand requires clustering to bind and activate Notch (Hicks et al., 2002, J. Neuroscience Research 68:655-667) and unclustered forms act as dominant negative inhibitors. In addition, Deltex can form homo-multimers indicating that clustering of Notch receptors may be necessary for function (Matsuno et al., 2002, Development 129:1049-1059). Thus, inhibiting the disassembly of Deltex mediated Notch oligomers may maintain the cells in an undifferentiated state. As Wnt is known to bind to the EGF-like repeats in regions that overlap with some of the Delta and Serrate binding sites (Wesley C, 1999, Mol Cell Biol. 19:5743-5758), Wnt binding at these sites may play a similar dominant negative role in disrupting CSL-independent Notch signaling. Wnt and Notch pathway interactions are common in both Drosophila and vertebrate developmental systems. For instance, antagonistic interactions have been found in human epidermal differentiation (Lowell et al., 2000, Curr. Biol. 10:491-500, Zhu A J and Watt F M, 1999, Development 126:2285-2298) and in mammary epithelial cell branching morphogenesis (uyttendaele et al., 1998, Dev. Biol. 196:204-217). This suggests that it may be possible to modulate Notch signaling by providing excess molecules comprising the extracellular domain of the Notch receptor to inhibit Wnt binding to Notch.

Together, these studies suggest that Notch acting through Deltex maintains an undifferentiated state and implicate Wnt signaling in disrupting this pathway. They also suggest that molecules that bind to the C-terminal region containing the PEST sequences may act as inhibitors of Notch signaling by blocking a disheveled mediated disruption of the signal. These molecules may include dominant negative portions of Dishevelled that bind this region but lack Notch signaling inhibition ability.

Experiments involving cell cultures indicate that the Deltex-Notch interaction prevents the cytoplasmic retention of Suppressor of Hairless/CSL protein family members, which are normally sequestered in the cytoplasm via association with the Notch ankyrin repeats and translocates to the nucleus when Notch binds to its ligand, Delta. On the basis of these findings Deltex appears to regulate Notch activity by antagonizing the interaction between Notch and Suppressor of Hairless/CSL protein family members. The translocation of the normally cytoplasmic Suppressor of Hairless/CSL protein family members to the nucleus when Notch binds to a ligand (Fortini & Artavanis-Tsakonas, 1994, Cell 79:273-282) is a convenient assay to monitor for Notch function as well as for the ability of Notch activators of the present invention to activate Notch function.

Suppressor of Hairless has been shown to be a DNA binding protein. Genetic and molecular data indicate that the activity of Suppressor of Hairless can be influenced by its binding to the nuclear protein Hairless. Moreover it appears that the transcription of at least some of the bHLH genes of the Enhancer of split complex depends directly on Notch signaling and the ability of Suppressor of Hairless/CSL protein family members to recognize the appropriate binding sites upstream of these genes. Manipulation of these various interactions (e.g., disrupting the interaction between Notch and Suppressor of Hairless/CSL protein family members with an antibody directed against the ankyrin repeats) will result in modulating the activity of the Notch pathway.

The Notch pathway can be manipulated by the binding of Notch ligands (e.g., Delta, Serrate) to the extracellular portion of the Notch receptor. Notch signaling appears to be triggered by the physical interaction between the extracellular domains of Notch and its membrane-bound ligands on adjacent cells. The expression of full-length ligands on one cell triggers the activation of the pathway in the neighboring cell that expresses the Notch receptor. Not surprisingly, the ligands act as activators of the pathway. On the other hand, the expression of truncated Delta or Serrate molecules that lack intracellular domains expressed in neighboring cells results in non-autonomous, dominant negative phenotypes. This demonstrates that these mutant forms of the receptor act as inhibitors of the pathway.

Notch signaling can also be modulated by altering the activity of the gamma-secretase complex, as described previously herein.

The Notch receptors can be modified and modulated by proteins belonging to the Fringe family or by O-fucosyltransferase-1 and proteins related to O-fucosyltransferases (Hicks et al. 2000, Nature Cell Biology 2, 515-520; Moloney et al. 2000, Nature 406, 369-375; Haltiwanger 2001, Trends in Glycoscience and Glycotechnology 13, 157-165; Panin et al. 2002, Journal of Biological Chemistry 277, 29945-29952; Okajima & Irvine 2002, Cell 111, 893-904). The Fringe protein family includes the Drosophila protein Fringe and the mammalian proteins Lunatic fringe, Manic fringe, and Radical fringe. Modulation of Fringe or O-fucosyltransferase expression or activity can alter the ligand specificity of Notch receptors or the level of Notch signaling (Hicks et al. 2000, Nature Cell Biology 2, 515-520; Moloney et al. 2000, Nature 406, 369-375; Okajima & Irvine 2002, Cell 111, 893-904). The Fringe/O-fucosyltransferase proteins can be overexpressed from expression vectors introduced into the embryonic stem cells by transfection or infection. The level of the Fringe/O-fucosyltransferase proteins can be modulated by antisense RNA, antisense oligonucleotides, antisense morpholino modified oligonucleotides or RNAi methodologies. The activity of the Fringe/O-fucosyltransferase proteins could also be modified by small molecule agonists and antagonists defined by bioassays.

The definition of the various molecular interactions among the Notch pathway elements provides additional specific pharmacological targets and assays that can be used to screen for Notch function activators and inhibitors. Having evaluated the consequences of a particular molecular manipulation in vivo, this information can be used to design biochemical in vitro screening assays for biological reagents or pharmaceuticals that interfere with or enhance Notch function.

Screening for molecules that trigger the dissociation of the Notch ankyrin repeats with Suppressor of Hairless/CSL family proteins and the subsequent translocation of Suppressor of Hairless/CSL family proteins from the cytoplasm to the nucleus can result in the identification of Notch activators. The activation of transcription of a reporter gene which has been engineered to carry several Suppressor of Hairless/CSL protein family members binding sites at its 5′ end in a cell that expresses Notch also results in the identification of activators of the pathway.

Reversing the underlying logic of these assays leads to the identification of inhibitors of Notch signaling. For example, cell lines expressing the aforementioned reporter gene can be treated with chemicals and biologicals and those which have the capacity to stop the expression of the reporter gene can be identified. The embryonic stem cell in which Notch function has been activated is subjected to cell growth conditions to induce proliferation. Such cell growth conditions (e.g., cell culture medium, temperature, if growth is done in vitro) can be any of those commonly known in the art. Preferably, both Notch activation and exposure to cell growth conditions is carried out in vitro. Contacting the cell with a Notch function activator and exposing the cell to cell growth conditions can be carried out concurrently or, if the activator acts over a sufficient period of time, sequentially (as long as Notch function activation to inhibit differentiation is present while cell growth occurs).

As used herein, the term “cell differentiation environment” refers to a cell culture condition wherein the pluripotent cells are induced to differentiate, or are induced to become a human cell culture enriched in differentiated cells. In one embodiment, the cell differentiation environment comprises a growth factor that induces differentiation. Preferably the cell lineage induced by the growth factor will be homogeneous in nature. The term “homogeneous,” refers to a population that contains more than 50%, 60%, 70%, 80%, 85%, 86%, 87%, 88%, 89%, 90%, 91%, 92%, 93%, 94%, 95%, 96%, 97%, 98%, or 99% of the desired cell lineage.

In accordance with the invention the medium of the cell differentiation environment may contain a variety of components including, for example, KODMEM medium (Knockout Dulbecco's Modified Eagle's Medium), DMEM, Ham's F12 medium, FBS (fetal bovine serum), FGF2 (fibroblast growth factor 2), KSR or hLIF (human leukemia inhibitory factor). The cell differentiation environment can also contain supplements such as L-Glutamine, NEAA (non-essential amino acids), P/S (penicillin/streptomycin), N2 and β-mercaptoethanol (β-ME). It is contemplated that additional factors may be added to the cell differentiation environment, including, but not limited to, fibronectin, laminin, heparin, heparin sulfate, retinoic acid, members of the epidermal growth factor family (EGFs), members of the fibroblast growth factor family (FGFs) including FGF2 and/or FGF8, members of the platelet derived growth factor family (PDGFs), transforming growth factor (TGF)/bone morphogenetic protein (BMP)/growth and differentiation factor (GDF) factor family antagonists including but not limited to noggin, follistatin, chordin, gremlin, cerberus/DAN family proteins, ventropin, and amnionless. TGF/BMP/GDF antagonists could also be added in the form of TGF/BMP/GDF receptor-Fc chimeras. Other factors that may be added include molecules that can activate or inactivate signaling through Notch receptor family, including but not limited to proteins of the Delta-like and Jagged families as well as inhibitors of Notch processing or cleavage. Other growth factors may include members of the insulin like growth factor family (IGF), the wingless related (WNT) factor family, and the hedgehog factor family. Additional factors may be added to promote neural stem/progenitor proliferation and survival as well as neuron survival and differentiation. These neurotrophic factors include but are not limited to nerve growth factor (NGF), brain derived neurotrophic factor (BDNF), neurotrophin-3 (NT-3), neurotrophin-4/5 (NT-4/5), interleukin-6 (IL-6), ciliary neurotrophic factor (CNTF), leukemia inhibitory factor (LIF), cardiotrophin, members of the transforming growth factor (TGF)/bone morphogenetic protein (BMP)/growth and differentiation factor (GDF) family, the glial derived neurotrophic factor (GDNF) family including but not limited to neurturin, neublastin/artemin, and persephin and factors related to and including hepatocyte growth factor.

In one embodiment, the differentiation medium contains no or very little serum. As used herein, “essentially serum free” refers to a medium that does not contain serum or serum replacement, or that contains essentially no or very little serum or serum replacement. As used herein, “essentially” means that a de minimus or reduced amount of a component, such as serum, may be present that does not eliminate the improved bioactive cell culturing capacity of the medium or environment. For example, essentially serum free medium or environment can contain less than 10, 9, 8, 7, 6, 5, 4, 3, 2, or 1% serum or serum replacement wherein the presently improved bioactive cell culturing capacity of the medium or environment is still observed. In one embodiment, the differentiating medium is a DMEM/F12 medium. In one embodiment of the present invention, the differentiating medium comprises a base salt solution. Preferably, the base salt solution is selected from the group consisting of DMEM, GMEM, and mixtures thereof.

In other embodiments, the cell differentiation environment comprises seeding an embryoid body to an adherent culture. As used herein, the terms “seeded” and “seeding” refer to any process that allows an embryoid body or a portion of an embryoid body to be grown in adherent culture. An used herein, the term “a portion” refers to at least one cell from an embryoid body, preferably between approximately 1-10 cells, more preferably between approximately 10-100 cells from an embryoid body, and more preferably still between approximately 50-1000 cells from an embryoid body. As used herein, the term “adherent culture” refers to a cell culture system whereby cells are cultured on a solid surface, which may in turn be coated with a substrate. The cells may or may not tightly adhere to the solid surface or to the substrate. The substrate for the adherent culture may further comprise any one or combination of polyornithine, laminin, poly-lysine, purified collagen, gelatin, extracellular matrix, fibronectin, tenascin, vitronectin, poly glycolytic acid (PGA), poly lactic acid (PLA), poly lactic-glycolic acid (PLGA) and feeder cell layers such as, but not limited to, primary astrocytes, astrocyte cell lines, glial cell lines, bone marrow stromal cells, primary fibroblasts or fibroblast cells lines. In addition, primary astrocyte/glial cells or cell lines derived from particular regions of the developing or adult brain or spinal cord including but not limited to olfactory bulb, neocortex, hippocampus, basal telencephalon/striatum, midbrain/mesencephalon, substantia nigra, cerebellum or hindbrain may be used to enhance the development of specific neural cell sub-lineages and neural phenotypes. Furthermore, the substrate for the adherent culture may comprise the extracellular matrix laid down by a feeder cell layer, or laid down by the pluripotent human cell or cell culture.

The cells produced using the methods of the present invention have a variety of uses. In particular, the cells may be used as a source of nuclear material for nuclear transfer techniques and used to produce cells, tissues or components of organs for transplant. The cells may be further differentiated into cells, such as, but not limited to, neural cells, that may be used as a source of nuclear material for nuclear transfer techniques and used to produce cells, tissues or components of organs for transplant. The neural cells can also be used in human cell therapy or human gene therapy to treat neuronal diseases such as Parkinson's disease, Huntington's disease, lysosomal storage diseases, multiple sclerosis, memory and behavioral disorders, Alzheimer's disease and macular degeneration. Other pathological conditions including stroke and spinal cord injury can be treated using the neural cells of the present invention. The neural cells can also be used in testing the effect of molecules on neural differentiation or survival, in toxicity testing or in testing molecules for their effects on neural or neuronal functions. This could include screens to identify factors with specific properties affecting neural or neuronal differentiation, development, survival or function. In this application the cell cultures could have great utility in the discovery, development and testing of new drugs and compounds that interact with and affect the biology of neural stem cells, neural progenitors or differentiated neural or neuronal cell types.

The term “feeder layer” is used interchangeably with the term “feeder cell layer”, includes a “feeder cell” and refers to a culture of cells that grows in vitro and secretes at least one factor into the culture medium, and that can be used to support the growth of another cell of interest in culture. As used herein, a “feeder cell layer” can be used interchangeably with the term “feeder cell.” A feeder cell can comprise a monolayer, where the feeder cells cover the surface of the culture dish with a complete layer before growing on top of each other, or can comprise clusters of cells. As used herein, the terms “cluster” and “clump” can be used interchangeably, and generally refer to a group of cells that have not been dissociated into single cells. The clusters may be dissociated into smaller clusters. This dissociation is typically manual in nature (such as using a Pasteur pipette), but other means of dissociation are contemplated. The cluster of cells can contain varying numbers of cells, ranging generally from 1 to 50,000 cells, more preferably from 1 to 10,000 cells, more preferably from 1 to 1000 cells, and most preferably from 100 to 1000 cells. Additionally, the cell of interest may or may not be cultured in direct contact with the feeder cell. For instance, the cell of interest can be co-cultured with the feeder cell in such a manner that the cell of interest is physically separated from the feeder cell by a membrane containing pores, yet the feeder cell still enriches the medium in such a way as to support the growth of the cell of interest.

Activating or inhibiting gamma-secretase or Notch signaling and function alone or while also activating or inhibiting other signaling or regulatory pathways could result in the maintenance of embryonic stem cells in a pluripotent state or could result in the controlled differentiation of the embryonic stem cells. In this way the pluripotent state could be stabilized by inhibiting the appropriate pathway(s) alone or by inhibiting the appropriate pathway(s) and inhibiting the appropriate differentiation signal or signals. Controlled stepwise differentiation could be accomplished by inhibiting gamma-secretase or Notch signaling or by the combination of inhibiting gamma-secretase or Notch signaling while activating appropriate differentiation pathways. Both approaches may be accomplished by using feeder layers that provide a niche in which the chosen combinations of signals are provided. For example, a feeder layer could be provided that expresses as a ligand an inhibitor of gamma-secretase or Notch signaling, or expresses an activator of Notch signaling. The entire ligand or only a portion of the ligand may be expressed in either a membrane bound or secreted form. The same feeder layer could also be engineered to express an activator or inhibitor of another signaling pathway, such as, for example, the Wnt signaling pathway. In all cases, any one or more of these factors may be provided in a soluble form, a membrane bound form or attached to substrates or extracellular matrix to immobilize them as necessary. In the context of controlled stepwise differentiation, a series of feeder layer niches could be used to guide cells down a particular differentiation pathway in a controlled manner.

It is also possible that the gamma-secretase or Notch ligands may act as either inhibitors or activators of gamma-secretase or Notch signaling, depending on the environment in which the cell is cultured. The activity of gamma-secretase or Notch ligands may depend on the other signaling pathways that are active in the embryonic stem cells. In addition, it is contemplated that although a ligand may act to stabilize a majority of the pluripotent cells in a cell culture, other pluripotent cells can differentiate in the presence of that same ligand. Similarly, it is contemplated that a ligand may act to differentiate a majority of pluripotent cells in a cell culture, other pluripotent cells can be stabilized in the presence of the same ligand.

As used herein, the terms “nucleic acid” and “polynucleotide” refer to RNA or DNA that is linear or branched, single or double stranded, or a hybrid thereof. The term also encompasses RNA/DNA hybrids. These terms also encompass untranslated sequence located at both the 3′ and 5′ ends of the coding region of the gene: at least about 1000 nucleotides of sequence upstream from the 5′ end of the coding region and at least about 200 nucleotides of sequence downstream from the 3′ end of the coding region of the gene. Less common bases, such as inosine, 5-methylcytosine, 6-methyladenine, hypoxanthine and others can also be used for antisense, dsRNA and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. The antisense polynucleotides and ribozymes can consist entirely of ribonucleotides, or can contain mixed ribonucleotides and deoxyribonucleotides. The polynucleotides of the invention may be produced by any means, including genomic preparations, cDNA preparations, in vitro synthesis, RT-PCR and in vitro or in vivo transcription.

An “isolated” nucleic acid molecule is one that is substantially separated from other nucleic acid molecules, which are present in the natural source of the nucleic acid (i.e., sequences encoding other polypeptides). Preferably, an “isolated” nucleic acid is free of some of the sequences that naturally flank the nucleic acid (i.e., sequences located at the 5′ and 3′ ends of the nucleic acid) in its naturally occurring replicon. For example, a cloned nucleic acid is considered isolated. In various embodiments, the isolated nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. A nucleic acid is also considered isolated if it has been altered by human intervention, or placed in a locus or location that is not its natural site, or if it is introduced into a cell by any method of transformation. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

Specifically excluded from the definition of “isolated nucleic acids” are: naturally-occurring chromosomes (such as chromosome spreads), artificial chromosome libraries, genomic libraries, and cDNA libraries that exist either as an in vitro nucleic acid preparation or as a transfected/transformed host cell preparation, wherein the host cells are either an in vitro heterogeneous preparation or plated as a heterogeneous population of single colonies. Also specifically excluded are the above libraries wherein a specified nucleic acid makes up less than 5% of the number of nucleic acid inserts in the vector molecules. Further specifically excluded are whole cell genomic DNA or whole cell RNA preparations (including whole cell preparations that are mechanically sheared or enzymatically digested). Even further specifically excluded are the whole cell preparations found as either an in vitro preparation or as a heterogeneous mixture separated by electrophoresis wherein the nucleic acid of the invention has not further been separated from the heterologous nucleic acids in the electrophoresis medium (e.g., further separating by excising a single band from a heterogeneous band population in an agarose gel or nylon blot).

The portion of the coding region of a gene can also encode a biologically active fragment of an protein. As used herein, the term “biologically active portion of” an protein is intended to include a portion, e.g., a domain/motif, of an protein that, when present in a cell culture of pluripotent cells stabilizes the cells in an pluripotent state. Typically, biologically active portions (e.g., peptides which are, for example, 5, 10, 15, 20, 30, 35, 36, 37, 38, 39, 40, 50, 100, or more amino acids in length) comprise a domain or motif with at least one activity of an compound that modulated the activity of gamma-secretase or Notch. Moreover, other biologically active portions in which other regions of the polypeptide are deleted, can be prepared by recombinant techniques and evaluated for one or more of the activities described herein.

The invention also provides chimeric or fusion polypeptides. As used herein, a “chimeric polypeptide” or “fusion polypeptide” comprises an gamma-secretase-modulating polypeptide operatively linked to a non-ganmma-secretase-modulating polypeptide, or comprises a Notch-modulating polypeptide operative linked to a non-Notch-modulating peptide. A “non-gamma-secretase-modulating polypeptide” refers to a polypeptide whose expression does not modulate the activity of the gamma-secretase signaling pathway. A “non-Notch-modulating polypeptide” refers to a polypeptide whose expression does not modulate the activity of the Notch signaling pathway. As used herein with respect to the fusion polypeptide, the term “operatively linked” is intended to indicate that the modulating polypeptide and the non-modulating polypeptide are fused to each other so that both sequences fulfill the proposed function attributed to the sequence used. The non-modulating polypeptide can be fused to the N-terminus or C-terminus of the modulating polypeptide. For example, in one embodiment, the fusion polypeptide is a GST-gamma-secretase-modulating fusion polypeptide in which the sequence of the gamma-secretase-modulating compound is fused to the C-terminus of the GST sequence. Such fusion polypeptides can facilitate the purification of recombinant polypeptides. In another embodiment, the fusion polypeptide is a Notch-modulating polypeptide containing a heterologous signal sequence at its N-terminus. In certain host cells (e.g., mammalian host cells), expression and/or secretion of a gamma-secretase-modulating or Notch-modulating polypeptide can be increased through use of a heterologous signal sequence.

An isolated nucleic acid molecule encoding a compound that modulates gamma-secretase or Notch can be created that has a certain percent sequence identity to a known polypeptide that modulates gamma-secretase activity by introducing one or more nucleotide substitutions, additions, or deletions into the known nucleotide sequence such that one or more amino acid substitutions, additions, or deletions are introduced into the encoded polypeptide. Mutations can be introduced by standard techniques, such as site-directed mutagenesis and PCR-mediated mutagenesis. Preferably, conservative amino acid substitutions are made at one or more predicted non-essential amino acid residues.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in the polypeptide is preferably replaced with another amino acid residue from the same side chain family. Alternatively, in another embodiment, mutations can be introduced randomly along all or part of a coding sequence, such as by saturation mutagenesis, and the resultant mutants can be screened for an ability to modulate gamma-secretase activity described herein to identify mutants that retain gamma-secretase-modulating activity. Following mutagenesis of the sequence, the encoded polypeptide can be expressed recombinantly and the activity of the polypeptide can be determined.

For the purposes of the invention, the percent sequence identity between two nucleic acid or polypeptide sequences may be determined using the “Blast Two Sequences” program available at National Center for Biotechnology Information (http://www.ncbi.nlm.nih.gov/blast/b12seq/b12.html). The percent sequence identity of two nucleic acids is determined using the algorithm of Karlin & Altschul, 1990 Proc. Natl. Acad. Sci. USA 87:2264-2268, modified as in Karlin & Altschul, 1993 Proc. Natl. Acad. Sci. USA 90:5873-5877. Such an algorithm is incorporated into the NBLAST and XBLAST programs of Altschul et al., 1990 J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al., 1997 Nucl. Acids. Res. 25:3389-3402. When utilizing BLAST and Gapped BLAST programs, the default parameters of the respective programs (NBLAST and XBLAST) are used. See http://www.ncbi.nih.gov/. It is to be understood that for the purposes of determining sequence identity when comparing a DNA sequence to an RNA sequence, a thymidine nucleotide is equivalent to a uracil nucleotide.

It is to be understood that for the purposes of determining sequence identity, when comparing a DNA sequence to an RNA sequence, a thymidine nucleotide is equivalent to a uracil nucleotide. Preferably, the isolated polypeptides are at least about 50-60%, preferably at least about 60-70%, and more preferably at least about 70-75%, 75-80%, 80-85%, 85-90%, or 90-95%, and most preferably at least about 96%, 97%, 98%, 99%, or more identical to a known entire amino acid sequence for a compound that modulates the activity of gamma-secretase or Notch.

Additionally, optimized nucleic acids can be created. In one embodiment, an optimized nucleic acid encodes an polypeptide that modulates gamma-secretase or Notch activity, and more preferably, the polypeptide acts to stabilize a pluripotent cell in culture. As used herein, “optimized” refers to a nucleic acid that is genetically engineered to increase its expression in a given animal. To provide optimized nucleic acids, the DNA sequence of the gene can be modified to 1) comprise codons preferred by highly expressed genes; 2) comprise an A+T content in nucleotide base composition to that substantially found in the animal; 3) form an initiation sequence, 4) eliminate sequences that cause destabilization, inappropriate polyadenylation, degradation and termination of RNA, or that form secondary structure hairpins or RNA splice sites. In addition, consideration is given to the percentage G+C content of the degenerate third base. Optimized nucleic acids of this invention also preferably have CG and TA doublet avoidance indices closely approximating those of the chosen host.

As used herein, “frequency of preferred codon usage” refers to the preference exhibited by a specific host cell in usage of nucleotide codons to specify a given amino acid. To determine the frequency of usage of a particular codon in a gene, the number of occurrences of that codon in the gene is divided by the total number of occurrences of all codons specifying the same amino acid in the gene. Similarly, the frequency of preferred codon usage exhibited by a host cell can be calculated by averaging frequency of preferred codon usage in a large number of genes expressed by the host cell. It is preferable that this analysis be limited to genes that are highly expressed by the host cell. The percent deviation of the frequency of preferred codon usage for a synthetic gene from that employed by a host cell is calculated first by determining the percent deviation of the frequency of usage of a single codon from that of the host cell followed by obtaining the average deviation over all codons. As defined herein, this calculation includes unique codons (i.e., ATG and TGG). In general terms, the overall average deviation of the codon usage of an optimized gene from that of a host cell is calculated using the equation 1A=n=1 Z Xn−Yn Xn times 100 Z where Xn=frequency of usage for codon n in the host cell; Yn=frequency of usage for codon n in the synthetic gene, n represents an individual codon that specifies an amino acid and the total number of codons is Z. The overall deviation of the frequency of codon usage, A, for all amino acids should preferably be less than about 25%, and more preferably less than about 10%. Preferably these indices deviate from that of the host by no more than about 10-15%.

In addition to the nucleic acid molecules encoding the polypeptides described above, another aspect of the invention pertains to isolated nucleic acid molecules that are antisense thereto. Antisense polynucleotides are thought to inhibit gene expression of a target polynucleotide by specifically binding the target polynucleotide and interfering with transcription, splicing, transport, translation and/or stability of the target polynucleotide. Methods are described in the prior art for targeting the antisense polynucleotide to the chromosomal DNA, to a primary RNA transcript or to a processed mRNA. Preferably, the target regions include splice sites, translation initiation codons, translation termination codons, and other sequences within the open reading frame.

The term “antisense,” for the purposes of the invention, refers to a nucleic acid comprising a polynucleotide that is sufficiently complementary to all or a portion of a gene, primary transcript, or processed mRNA, so as to interfere with expression of the endogenous gene. “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. The term “antisense nucleic acid” includes single stranded RNA as well as double-stranded DNA expression cassettes that can be transcribed to produce an antisense RNA.

In addition to the nucleic acids and polypeptides described above, the present invention encompasses these nucleic acids and polypeptides attached to a moiety. These moieties include, but are not limited to, detection moieties, hybridization moieties, purification moieties, delivery moieties, reaction moieties, binding moieties, and the like. A typical group of nucleic acids having moieties attached are probes and primers. Probes and primers typically comprise a substantially isolated oligonucleotide. The oligonucleotide typically comprises a region of nucleotide sequence that hybridizes under stringent conditions to at least about 12, preferably about 25, more preferably about 40, 50, or 75 consecutive nucleotides of a sense strand of the nucleic acid sequences. Primers can be used in PCR reactions to clone homologs of the known polypeptides that modulate the activity of gamma-secretase or Notch. Probes based on nucleotide sequences can be used to detect transcripts or genomic sequences encoding the same or homologous polypeptides. In preferred embodiments, the probe further comprises a label group attached thereto, e.g. the label group can be a radioisotope, a fluorescent compound, an enzyme, or an enzyme co-factor. Such probes can be used as a part of a genomic marker test kit for identifying cells which express a compound that modulates gamma-secretase or Notch activity.

The invention further provides an isolated recombinant expression vector comprising a nucleic acid, wherein expression of the vector in a host cell results in increased modulation of gamma-secretase or Notch compared to a wild-type variety of the host cell. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses, and adeno-associated viruses), which serve equivalent functions.

In one embodiment, a gamma-secretase-modulating or Notch-modulating protein is expressed in mammalian cells using a mammalian expression vector. Examples of mammalian expression vectors include pCDM8 (Seed, B., 1987, Nature 329:840) and pMT2PC (Kaufinan et al., 1987, EMBO J. 6:187-195). When used in mammalian cells, the expression vector's control functions are often provided by viral regulatory elements. For example, commonly used promoters are derived from polyoma, Adenovirus 2, cytomegalovirus, and Simian Virus 40. For other suitable expression systems for both prokaryotic and eukaryotic cells, see chapters 16 and 17 of Sambrook, J., Fritsh, E. F., and Maniatis, T. Molecular Cloning: A Laboratory Manual, 2nd Ed., Cold Spring Harbor Laboratory, Cold Spring Harbor Laboratory Press, Cold Spring Harbor, N.Y., 1989.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. As used herein, the term “selectable marker” refers to a gene encoding a protein necessary for the survival or growth of a host cell transformed with the vector. Although such a marker gene may be carried on another polynucleotide sequence co-introduced into the host cell, it is most often contained on the cloning vector. Only those host cells into which the marker gene has been introduced will survive and/or grow under selective conditions. Typical selection genes encode proteins that (a) confer resistance to antibiotics or other toxic substances, e.g., ampicillin, neomycin, methotrexate, etc.; (b) complement auxotrophic deficiencies; or (c) supply critical nutrients not available from complex media. The choice of the proper selectable marker will depend on the host cell; appropriate markers for different hosts are known in the art.

Other suitable methods for transforming or transfecting host cells can be found in Sambrook, et al, Molecular Cloning: A Laboratory Manual. 2nd Ed., Cold Spring Harbor Laboratory, Cold Spring Harbor Laboratory Press, Cold Spring Harbor, N.Y., 1989, and other laboratory manuals.

In another embodiment, recombinant organisms can be produced that contain selected systems which allow for regulated expression of the introduced gene. For example, inclusion of a gene encoding a gamma-secretase-modulating protein or Notch-modulating protein on a vector placing it under control of the lac operon permits expression of the gene encoding the protein only in the presence of IPTG. Such regulatory systems are well known in the art.

Gene expression should be operatively linked to an appropriate promoter conferring gene expression in a timely, cell or tissue specific manner. Promoters useful in the expression cassettes of the invention include any promoter that is capable of initiating transcription in a cell.

The promoter may be constitutive, inducible, developmental stage-preferred, cell type-preferred, tissue-preferred, or organ-preferred. Constitutive promoters are active under most conditions. Examples of constitutive promoters include the CaMV 19S and 35 S promoters (Odell et al., 1985, Nature 313:810-812), the sX CaMV 35S promoter (Kay et al., 1987, Science 236:1299-1302) the Sep1 promoter, the ubiquitin promoter (Christensen et al., 1989, Plant Molec Biol 18:675-689); pEmu (Last et al., 1991, Theor. Appl. Genet. 81:581-588), and the like.

Inducible promoters are active under certain environmental conditions, such as the presence or absence of a nutrient or metabolite, heat or cold, light, pathogen attack, anaerobic conditions, and the like. Chemically inducible promoters are especially suitable if gene expression is wanted to occur in a time specific manner. Developmental stage-preferred promoters are preferentially expressed at certain stages of development Additional flexibility in controlling heterologous gene expression may be obtained by using DNA binding domains and response elements from heterologous sources (i.e., DNA binding domains from non-mammalian sources).

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. With respect to a recombinant expression vector, “operatively linked” is intended to mean that the nucleotide sequence of interest is linked to the regulatory sequence(s) in a manner which allows for expression of the nucleotide sequence (e.g., in an iii vitro transcription/translation system or in a host cell when the vector is introduced into the host cell). The term “regulatory sequence” is intended to include promoters, enhancers, and other expression control elements (e.g., polyadenylation signals). Such regulatory sequences are described, for example, in Goeddel, Gene Expression Technology: Methods in Enzymology 185, Academic Press, San Diego, Calif. (1990) and Thompson, Chapter 7, 89-108, CRC Press: Boca Raton, Fla., including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides or peptides, encoded by nucleic acids as described herein (e.g., gamma-secretase-modulating polypeptides, Notch-modulating polypeptides, fusion polypeptides, etc.).

As used herein, “recombinant host cells” are those which have been genetically modified to contain an isolated or other recombinant DNA molecule, as described herein. The DNA can be introduced by any means known to the art which is appropriate for the particular type of cell, including without limitation, transformation with plasmids, including different methods of plasmid delivery such as, without limitation, liposomal delivery, electroporation, or naked plasmid injection; transduction with viral vectors; or DNA delivery mediated by polymeric agents.

In addition to fragments and fusion polypeptides of the gamma-secretase-modulating and Notch-modulating proteins described herein, the present invention includes homologs and analogs of naturally occurring gamma-secretase-modulating proteins and nucleotides encoding gamma-secretase-modulating proteins, and of naturally occurring Notch-modulating proteins and nucleotides encoding Notch-modulating proteins. “Homologs” are defined herein as two nucleic acids or polypeptides that have similar, or substantially identical, nucleotide or amino acid sequences, respectively. Homologs include allelic variants, orthologs, paralogs, agonists and antagonists of proteins as defined hereafter. The term “homolog” further encompasses nucleic acid molecules that differ due to degeneracy of the genetic code and thus encode the same protein molecule. As used herein a “naturally occurring” protein refers to a protein amino acid sequence that occurs in nature. Similarly, a “naturally occurring” isolated nucleotide encoding a gamma-secretase-modulating or Notch-modulating protein refers to a nucleic acid sequence that occurs in nature.

Nucleic acid molecules corresponding to natural allelic variants and analogs, orthologs and paralogs of a protein or isolated nucleotide encoding a protein can be isolated using a hybridization probe according to standard hybridization techniques under stringent or moderate hybridization conditions. In an alternative embodiment, homologs can be identified by screening combinatorial libraries of mutants, for agonist or antagonist activity. There are a variety of methods that can be used to produce libraries of potential protein homologs from a degenerate oligonucleotide sequence. Chemical synthesis of a degenerate gene sequence can be performed in an automatic DNA synthesizer, and the synthetic gene is then ligated into an appropriate expression vector. Use of a degenerate set of genes allows for the provision, in one mixture, of all of the sequences encoding the desired set of potential protein sequences. Methods for synthesizing degenerate oligonucleotides are known in the art. See, e.g., Narang, S. A., 1983, Tetrahedron 39:3; Itakura et al., 1984, Annu. Rev. Biochem. 53:323; Itakura et al., 1984, Science 198:1056; Ike et al., 1983, Nucleic Acid Res. 11:477.

In addition, libraries of fragments of protein coding regions can be used to generate a variegated population of protein fragments for screening and subsequent selection of homologs of a gamma-secretase-modulating or Notch-modulating protein. In one embodiment, a library of coding sequence fragments can be generated by treating a double stranded PCR fragment of a gamma-secretase-modulating or Notch-modulating protein coding sequence with a nuclease under conditions wherein nicking occurs only about once per molecule, denaturing the double stranded DNA, renaturing the DNA to form double stranded DNA, which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal, and internal fragments of various sizes of the gamma-secretase-modulating or Notch-modulating protein.

Hybridization procedures are useful for identifying polynucleotides with sufficient homology to the subject regulatory sequences to be useful as taught herein. The particular hybridization technique is not essential to the subject invention. As improvements are made in hybridization techniques, they can be readily applied by one of ordinary skill in the art.

Various degrees of stringency of hybridization can be employed for studies of cloned sequences isolated as described herein. The more stringent the conditions, the greater the complementarity that is required for duplex formation. Stringency can be controlled by temperature, probe concentration, probe length, ionic strength, time, and the like. Preferably, hybridization is conducted under moderate to high stringency conditions by techniques well known in the art, as described, for example in Keller, G. H., M. M. Manak, 1987 DNA Probes, Stockton Press, New York, N.Y., pp. 169-170, hereby incorporated by reference. In a preferred embodiment, the hybridization is selective for target DNA. As used herein, the term “selective hybridization” or “selectively hybridizing” refers to the ability to discern between the binding of a nucleic acid sequence to a target DNA sequence as compared to other non-target DNA sequences.

As used herein, moderate to high stringency conditions for hybridization are conditions that achieve the same, or about the same, degree of specificity of hybridization as the conditions described herein. As used herein, the term “highly stringent ” or “high stringency conditions” comprises hybridizing at 68° C. in 5× SSC/5× Denhardt's solution/0.1% SDS, and washing in 0.2× SSC/0.1% SDS at 65° C. As used herein, the term “moderately stringent” or “moderate stringency conditions” comprise hybridizing at 55C in 5× SSC/5× Denhardt's solution/0.1% SDS and washing at 42° C. in 3× SSC. The parameters of temperature and salt concentration can be varied to achieve the desired level of sequence identity between probe and target nucleic acid. See, e.g., Sambrook et al., 1989 Molecular Cloning, Second Edition, Cold Spring Harbor Laboratory, Plainview, N.Y. Ausubel et al., 1995 Current Protocols in Molecular Biology, John Wiley & Sons, NY, N.Y., Meinkoth and Wahl, 1984, Anal. Biochem. 138:267-284; or Tijssen, 1993, Laboratory Techniques in Biochemistry and Molecular Biology: Hybridization with Nucleic Acid Probes, Part I, Chapter 2, Elsevier, N.Y., for further guidance on hybridization conditions.

One subset of these homologs is allelic variants. As used herein, the term “allelic variant” refers to a nucleotide sequence containing polymorphisms that lead to changes in the amino acid sequences of a protein and that exist within a natural population. Such natural allelic variations can typically result in 1-5% variance in a protein or isolated nucleotide encoding a protein.

Moreover, nucleic acid molecules encoding proteins from the same or other species such as analogs, orthologs, and paralogs, are intended to be within the scope of the present invention. As used herein, the term “analogs” refers to two nucleic acid sequences that have the same or similar function, but that have evolved separately in unrelated organisms. As used herein, the term “orthologs” refers to two nucleic acids from different species, but that have evolved from a common ancestral gene by speciation. Normally, orthologs encode polypeptides having the same or similar functions. As also used herein, the term “paralogs” refers to two nucleic acids that are related by duplication within a genome. Paralogs usually have different functions, but these functions may be related (Tatusov, R. L. et al., 1997, Science 278(5338):631-637). Analogs, orthologs and paralogs of a naturally occurring protein can differ from the naturally occurring protein by post-translational modifications, by amino acid sequence differences, or by both. Post-translational modifications include in vivo and in vitro chemical derivatization of polypeptides, e.g., acetylation, carboxylation, phosphorylation, or glycosylation, and such modifications may occur during polypeptide synthesis or processing or following treatment with isolated modifying enzymes. In particular, orthologs of the invention will generally exhibit at least 80-85%, more preferably, 85-90% or 90-95%, and most preferably 95%, 96%, 97%, 98% or even 99% identity or sequence identity with all or part of a naturally occurring protein amino acid sequence and will exhibit a function similar to that protein.

A host cell of the invention, such as a eukaryotic host cell in culture, can be used to produce (i.e., express) a protein. Accordingly, the invention further provides methods for producing proteins using the host cells of the invention. In one embodiment, the method comprises culturing the host cell of invention (into which a recombinant expression vector encoding a target protein has been introduced, or into which genome has been introduced a gene encoding a wild-type or altered target protein) in a suitable medium until the target protein is produced. In another embodiment, the method encompasses the introduction of a heterologous isolated nucleotide encoding a target protein, resulting in a down-regulation in secretion of the target protein. It is contemplated that the host cell can be a pluripotent cell or a feeder cell.

The present invention farther encompasses the use of gamma-secretase complex or Notch expressing pluripotent cells to identify a compound that modulates the pluripotency of said cells. Such a method comprises (a) contacting pluripotent cells that express at least one component of the gamma-secretase complex or one or more Notch proteins with a test compound; and (b) determining the effect of the test compound on the pluripotency of the pluripotent cells, the test compound being identified as a modulator of pluripotency based on the ability of the test compound to modulate the pluripotency of the pluripotent cells. Pluripotency of cells can be determined using methods well known to those of skill in the art, and can include, for example, examination of cell morphology, and analysis of mRNA and protein levels.

As used herein, the term “test compound” is intended to refer to a compound that has not previously been identified as, or recognized to be, a modulator of gamma-secretase activity, a modulator or Notch activity, or of pluripotency.

The definition of the various molecular interactions among the gamma-secretase pathway elements provides additional specific pharmacological targets and assays that can be used to screen for activators and inhibitors of gamma-secretase or Notch. Having evaluated the consequences of a particular molecular manipulation in vivo, this information can be used to design biochemical in vitro screening assays for biological reagents or pharmaceuticals that interfere with or enhance gamma-secretase or Notch function.

Standard techniques for cloning, DNA isolation, amplification and purification, for enzymatic reactions involving DNA ligase, DNA polymerase, restriction endonucleases and the like, various separation techniques, and techniques to analyze mRNA and proteins are those known and commonly employed by those skilled in the art. A number of standard techniques are described in Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, New York; Wu (Ed.) 1993 Meth. Enzymol. 218, Part I; Wu (Ed.) 1979 Meth. Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of Calif. Press, Berkeley; Schleif and Wensink, 1982 Practical Methods in Molecular Biology; Glover (Ed.) 1985 DNA Cloning Vol. I and II, IRL Press, Oxford, UK; Hames & Higgins (Eds.) 1985 Nucleic Acid Hybridization, IRL Press, Oxford, UK; and Setlow & Hollaender 1979 Genetic Engineering: Principles and Methods, Vols. 1-4, Plenum Press, N.Y. Abbreviations and nomenclature, where employed, are deemed standard in the field and commonly used in professional journals such as those cited herein.

Throughout this application, various publications are referenced. The disclosures of all of these publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments.

### EXAMPLES

### Example 1

Notch1 is a Marker for Undifferentiated Human ES Cells and is Down-Regulated upon Differentiation

Materials and Methods

Antibodies

Antibodies to SSEA1, SSEA3, SSEA4, and bTAN20 (Notch1) were all from Developmental Studies Hybridoma Bank. Notch-1(H-131) is a rabbit polyclonal antibody from Santa Cruz Technologies. Antibodies to TRA-1-60 and TRA-1-81 were a gift from Peter Andrews.

Immunohistochemistry

Cells on chamber slides were rinsed once with 1× PBS and fixed for 30 minutes in 4% PFA/4% sucrose in PBS pH7.4 at room temperature for surface staining, or in ice cold 100% methanol for 5 minutes at −20° C. followed by 4% PFA/4% sucrose in PBS for 10 minutes at room temperature for intracellular staining using the bTAN20 antibody. They were rinsed 3× in 1×PBS and blocked in 3% goat serum/1% PVP with or without 0.3% Triton-X100 in PBS for 30 minutes at 4° C. Primary antibodies were diluted in blocking solution and this solution was applied overnight at 4° C. to the cells. Cells were rinsed in 1×PBS and washed overnight with 3 changes of 1×PBS. Secondary antibodies were applied in blocking solution for 2 hours at 4° C. They were rinsed in 1×PBS then washed overnight with 3 changes of 1×PBS. Cell nuclei were stained with DAPI at 1 μg/ml in the first rinse for 10 minutes. The chambers were removed and slides were mounted in VectaShield mounting medium.

RNA Preparation and RT-PCR

Total RNA was extracted from cell samples with Trizol reagent (Cat# 15596-026, Invitrogen, Carlsbad, Calif.). Reverse transcription (RT) reactions were performed with oligo-dT using a kit (Cat. # 11904-018, Invitrogen). PCR reactions of 50 μl containing 2 μl of cDNA, 1× PCR buffer, 1.5 MM MgCl₂, 1 mM dNTP mix, 0.5 μM of each specific primer and 2 units of TAQ. Primers for Deltex (accession # AF053700) were selected to span an intron using Biology Workbench 3.2. The primers used were: Deltex forward 5′-GTGCCCTACATCATCGACCT-3′ (SEQ ID NO:1), reverse 5′-CTGCGACATGCTGTTGAAGT-3′ (SEQ ID NO:2). Other specific primers used were: Notch1 forward 5′-GATGCCAACATCCAGGACAACATGGG-3′ (SEQ ID NO:3); reverse 5′-GGCAGGCGGTCCATATGATCCGTGAT-3′ (SEQ ID NO:4); Notch2 forward 5′-ACATCATCACAGACTTGGTC-3′ (SEQ ID NO:5); reverse 5′-CATTATTGACAGCAGCTGCC-3′ (SEQ ID NO:6) (Karanu et al. 2000, J. Exp. Med. 192:1365-1372); Jagged1 forward 5′-ACACACCTGAAGGGGTGCGGTATA-3′ (SEQ ID NO:7); reverse 5′-AGGGCTGCAGTCATTGGTATTCTGA-3′ (SEQ ID NO:8); Jagged2 forward 5′-CAGTGGCTTTACTGGCACCTACTGC-3′ (SEQ ID NO:9), reverse 5′-GGGTTGCAGTCGTTGGTATTGTGAG-3′ (SEQ ID NO:10); Delta forward 5′-TGCTGGGCGTCGACTCCTTCAGT-3′ (SEQ ID NO:11), reverse 5′-GCCTGGATAGCGGATACACTCGTCACA-3′ (SEQ ID NO:12) (Ignatova et al., 2002, Glia 39:193-206).

Forty cycles of amplification were done for Deltex and 30 cycles for the other genes. Each three-step cycle was 94° for 45 seconds, 60° for 45 seconds and 72° for 30 seconds.

Cell Culture

Feeder Cell Lines

BGN1 hES cells maintained on mouse embryonic fibroblasts (MEFs) were passaged by manually breaking up colonies using fire-drawn Pasteur pipettes and replating the cell clumps on fresh MEFs. Alternatively BGN1 hES cells were passaged with a combination of Collagenase treatment and trypsinization. In this case, colonies were treated with 1 mg/ml Collagenase (Gibco) in growth medium consisting of 20% KSR/1% MEM nonessential amino acids/1 mM L-glutamine/penicillin (0.5 U/ml) and streptomycin (0.5 U/ml)/0.1 mM beta-mercaptoethanol for 3-5 minutes on a warm stage. The Collagenase was removed and 0.05% trypsin/EDTA (Gibco) was added. The colonies were immediately flushed off of the MEF feeder layer and triturated to a single cell suspension. The cells were not in the trypsin solution for more than approximately 45 seconds. The trypsin was neutralized with 10% FBS/10% KSR human ES medium. The cells were washed in growth medium, counted, and plated at a density of 20,000 cells/cm² on MEFs.

Magnetic Sorting

Collagenase/trypsin passaged cells were enriched for SSEA4 expressing cells with the MACS magnetic cell sorting system (Miltenyi Biotec, Inc.). Manually passaged HESCs were harvested by treating with 1 mg/ml Collagenase (Gibco) for 5 minutes, followed by 0.05% Trypsin/EDTA for 30 seconds. Colonies were then flushed off the top of the feeder layer and dissociated to a single cell suspension, leaving the feeders behind as a net. The trypsin was neutralized with 10% FBS/10% KSR human ES medium and passed through a cell strainer (Becton, Dickinson). For blocking, cells were pelleted and resuspended in cold staining buffer (5% FBS, 1 mM EDTA, penicillin (0.5 U/ml) and streptomycin (0.5 U/ml), in Ca/Mg-free PBS). The cells were pelleted and resuspended in 1 ml primary antibody (anti-SSEA4, Developmental Studies Hybridoma Bank) diluted 1:10 in staining buffer, at 4° C. for 15 minutes. 9 ml staining buffer was then added and the cells were pelleted, washed with 10 ml staining buffer and repelleted. 1×10⁷ cells were resuspended in 80 staining buffer and magnetic goat anti-mouse IgG MicroBeads (Miltenyi Biotec, Inc) were added, mixed and incubated at 4° C. for 10 minutes. The volume was then brought to 2 ml with staining buffer and 1 μl of a fluorescent conjugated secondary antibody (Alexa-488 conjugated goat anti-mouse IgG, Molecular Probes) was added to enable analysis of the success of the separation. The sample was incubated for 5 minutes at 4° C., then the volume was brought to 10 ml with staining buffer and the cells were pelleted and washed in 10 ml staining buffer and repelleted. The cells were resuspended in 500 μl staining buffer and applied to a separation column, that had been prepared by washing three times with 500 μl staining buffer. The column was positioned on the selection magnet prior to application of the cells, and the flow through and three washes with 500 μl staining buffer were collected (presumed SSEA4 negative population). The column was removed from the magnet, 500 μl staining buffer was added, forced through with a plunger and the presumed SSEA4 positive cell population collected in a 15 ml tube. 20% KSR human ES growth medium was added to 10 ml, the cells were pelleted and resuspended in 1 ml of the same medium. 10⁵ SSEA4 selected HESCs were plated on 35 mm MEF dishes and maintained and passaged in 20% KSR growth medium. To examine the effectiveness of the selection, aliquots of the flow/wash sample and SSEA4 selected sample were analyzed by fluorescence microscopy. Approximately 75% of the cells from the retained fraction were SSEA4 positive, indicating effective enrichment.

Results

Notch1 is a marker for undifferentiated human ES cells and is quickly down regulated upon differentiation.

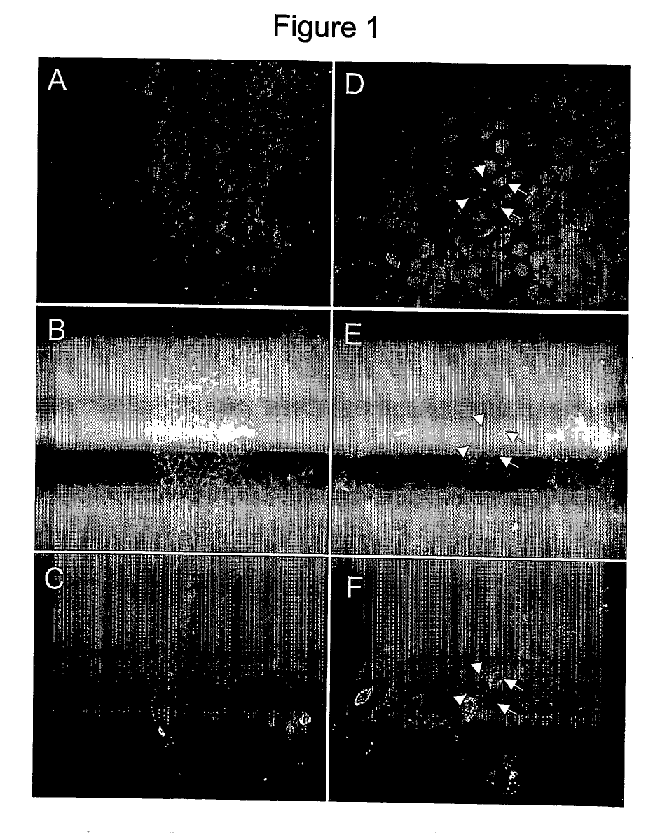

Notch 1 is highly expressed on the surface of morphologically undifferentiated hES cells (FIG. 1B). These cells also express SSEA4 (FIG. 1C). In differentiating regions of manually passaged human ES colony differentiating cells are negative for Notch-1 (FIG. 1E, arrowheads) and SSEA4 (FIG. 1F, arrowheads). These cells can be seen adjacent to cells that are still positive for Notch1 (FIG. 1E, arrows) and SSEA4 (FIG. 1F, arrows).

SSEA4 Selection of Trypsin Passaged BGN1 hES Cells

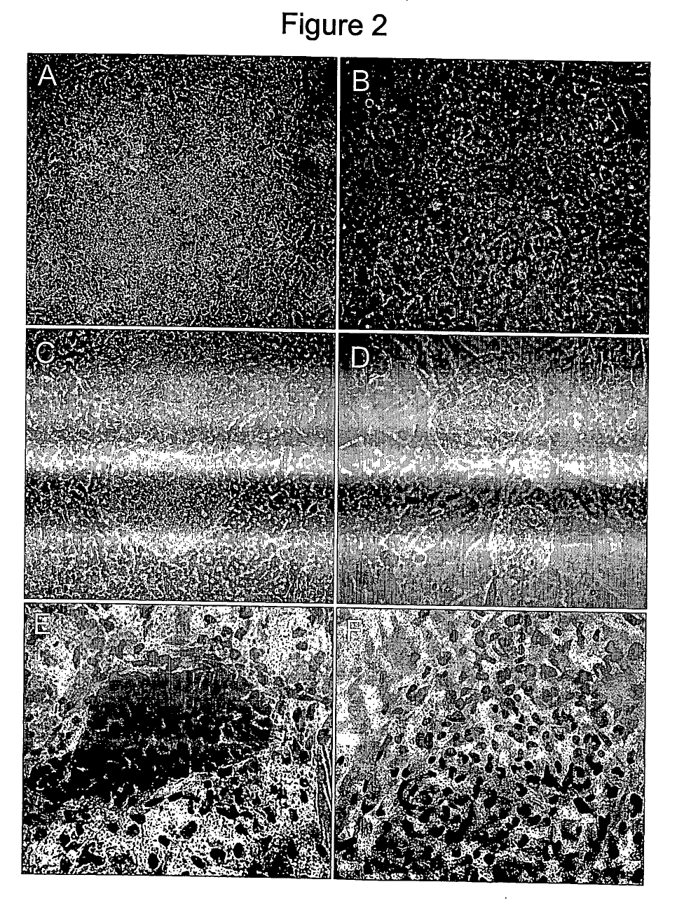

In order to derive stable expandable lines of hES cells from the manually passaged cell lines, trypsin was used to passage manual colonies as single cells. Five colonies with good cellular morphology were chosen for trypsin passaging. After two passages the colonies grew without well defined borders with cells at the edge integrating into the feeder layer (FIG. 2A). However, at higher magnification of A (FIG. 2B), the cells in the center of the colony maintain a morphology similar to that of manually passaged hES cells. These cells were small with a large nucleus and had distinct borders. In addition the extracellular space was well defined.

At passage 8, in addition to the first colony type, a small number of colonies with a compact dome morphology appeared (FIG. 2C). These cells were also small with a large round nucleus. Spaces between cells in the colony were also well defined. Magnetic sorting for SSEA4 expression enriched for the compact dome colony morphology (FIG. 2D). 42% of colonies that grew from the retained fraction had a compact dome morphology, whereas 8% of colonies from the flow-through had this morphology. Oct4 staining of a colony from the retained fraction (FIG. 42E) from the SSEA4 magnetic sorting procedure shows the nuclear morphology of the compact dome colonies. The majority of the colonies in the flow-through had a flat morphology. Individual cells in these colonies were positive for Oct4 (FIG. 2F), weakly positive for SSEA4 and often contained U-shaped nuclei. The compact dome colony morphology has been stabilized for more than 20 passages. These cells can be frozen and recovered.

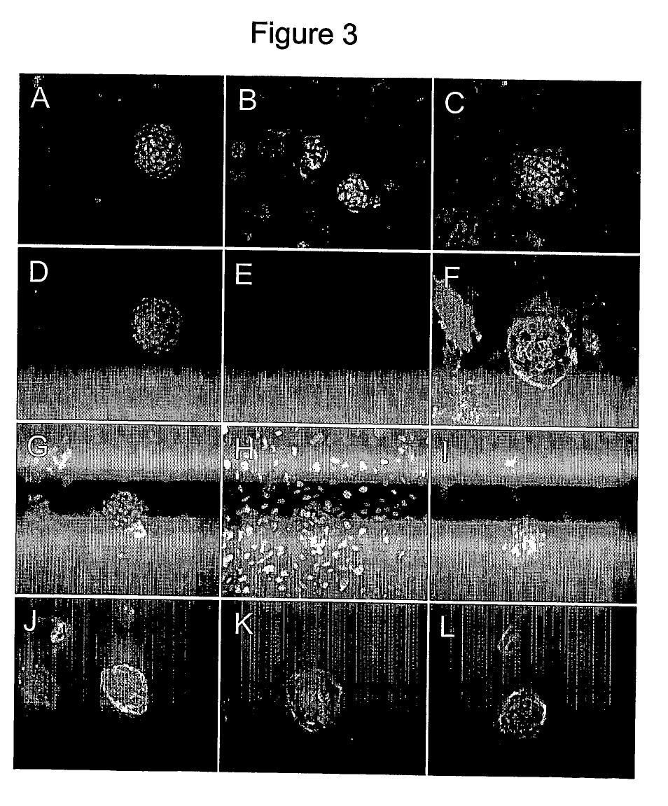

SSEA4 sorted cells express markers of pluripotent hES cells including Oct4 (FIG. 3D), SSEA3 (FIG. 3F), SSEA4 (FIG. 3J), TRA-1-60 (FIG. 3K), and TRA-1-81 (FIG. 3L). Similarly to hES cells, they do not express SSEA1 (FIG. 3E),

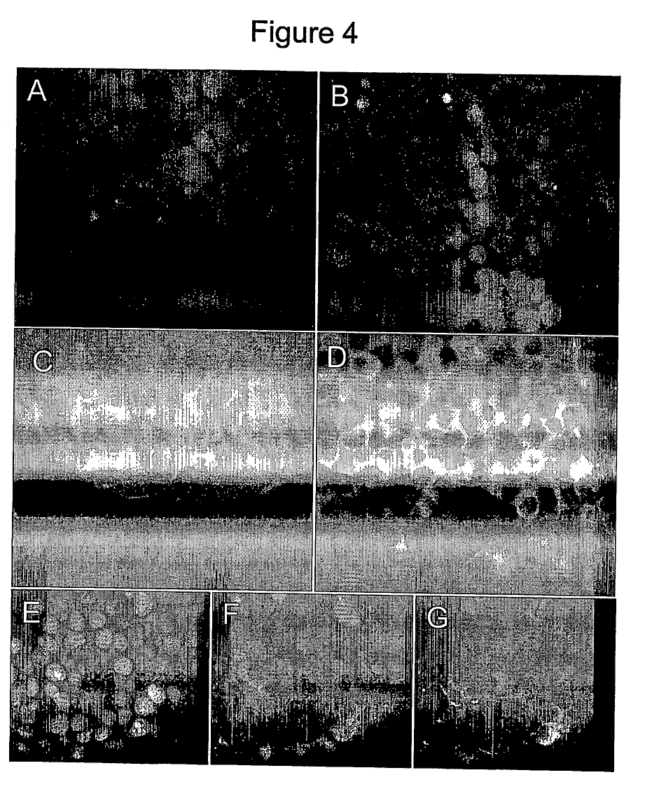

In cells that have been passaged as single cells and enriched for cells expressing SSEA4, Notch-1 is still expressed. These undifferentiated SSEA4 selected BGN1 hES cells are uniformly stained with an antibody recognizing the intracellular domain of Notch-1 (FIG. 4C) as well as with an antibody recognizing an extracellular epitope of Notch1 (FIG. 4D). In addition, as the colonies begin spontaneous differentiation they lose surface staining as shown by staining with an antibody that recognizes an extracellular epitope (FIG. 4D). Cells at the edge of the colony (bottom of FIG. 4D) rapidly lose surface expression of Notch-1. Differentiating cells that lose Notch-1 expression also lose SSEA4 expression (FIG. 4E-G). Thus, Notch-1 is expressed in both manually passaged hES cells and SSEA4 selected trypsin passaged cells and is down-regulated in differentiating cells.

Notch Signaling Appears to be Active in hES Cells

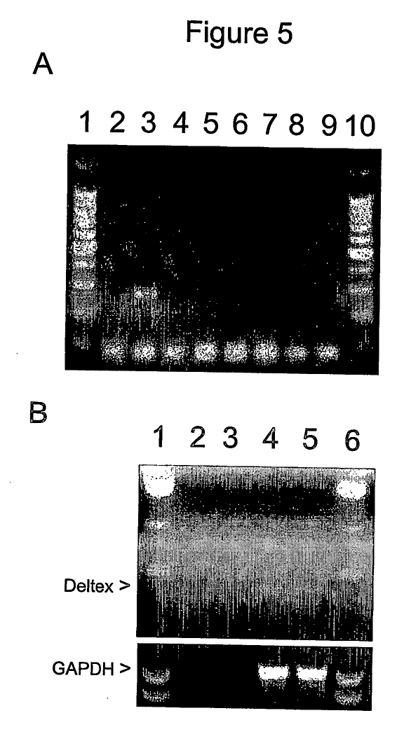

Thus far, the evidence for Notch activation is Deltex. In genetic screens in Drosophila, Deltex has been shown to act as a positive regulator of Notch signaling. In some cases Deltex has been shown to be activated by Notch activity. However, its expression has also been shown to be independently regulated. Deltex is expressed in both manually passaged (FIG. 5B, lane 5) and SSEA4 selected trypsin passaged cells (FIG. 5A, lane 3 and 5B, lane 4). In comparison, human fibroblast and stromal cells lines, which express Notch-1 and 2 (Table 1), do not express Deltex (FIG. 5A, lanes 5-9). Therefore, the presence of Deltex in the BGN1 selected hES cells is suggestive of Notch activation.

Human fibroblast and stromal cell lines that were either capable or incapable of supporting manual passage human ES cells in a pluripotent state were screened for expression of Notch ligands and receptors to determine if there was a correlation between the ability of the lines to support human ES cell growth (HS27 and KEL, Table 1) and ligand expression. By RT-PCR for expression of Jagged1 and 2, and Delta, there was not a clear trend (Table 1). At least one ligand, Jagged1, was expressed in all cell lines tested. However, it is still possible that presence of protein is not reflected in mRNA expression.

TABLE 1
Summary of RT-PCR results comparing Notch receptor and
ligand expression on SSEA4 selected BGN1 hES cells and
human feeder cell lines.

| | BGN1 | BJ | HS27A | Huvec | Jeg | KeIFib | WS1 |
|---|---|---|---|---|---|---|---|
| Deltex | +/− | − | − | − | − | − | − |
| GAPDH | + | + | + | + | + | + | + |
| Notch 1 | + | + | + | + | + | + | + |
| Notch 2 | + | + | + | + | + | + | + |
| Jagged 1 | + | + | + | + | + | + | + |
| Jagged 1 | + | + | + | + | + | + | + |
| Jagged 2 | + | − | − | + | +/− | − | + |
| Jagged 2 | + | +/− | +/− | + | +/− | +/− | + |
| Delta | ND | ND | + | − | +/− | +/− | + |
| Delta | + | +/− | − | − | − | − | + |
| Delta | + | + | + | − | +/− | +/− | + |

### Example 2

Heterogeneity of Manual and Trypsin Passaged HESCs

Materials and Methods

Antibodies

Antibodies to SSEA1, SSEA3, SSEA4, Notch1 (H-131), bTAN20 (Notch 1), TRA-1-60, and TRA-1-81 were obtained as described in Example 1. Antibodies to C651.6DbHN (Notch-2) were from Developmental Studies Hybridoma Bank. Antibodies to Oct-4 (catalog No. sc-5279) were from Santa Cruz Technologies. Presenilin-1 (catalog No. MAB5232) and Nicastrin (catalog No. AB5890) antibodies were from Chemicon International, Inc. The antibody to HDAC2 was from Zymed Laboratories, Inc. The Cleaved Notch1 antibody (NICD; catalog No. 2421) was from Cell Signaling Technology, Inc. Secondary Alex Fluor conjugated antibodies were from Molecular Probes, Inc.

Immunohistochemistry

Immunohistochemistry was performed as described in Example 1.

Western Blots

Western blots were done using standard procedures. In brief, protein content of samples was determined with a BCA micro protein assay (Pierce, Rockford, Ill.) and equal amounts of protein were loaded onto precast polyacrylamide mini-gels (Gradipore, Australia). Proteins were transferred to nylon membranes which were then incubated for 30 minutes in blocking buffer (tris buffered saline/0.1% tween 20/5% powdered milk). Primary antibodies were diluted in blocking buffer at the following ratios: bTAN20 and C651.6bHN were diluted 1:10; anti-Oct4 was diluted 1:100; anti-presenilin, nicastrin, cleaved NOTCH1 were diluted 1:1000 and anti-HDAC was used at a concentration of 0.5 μg/ml. Primary antibodies were incubated overnight at 4° C., followed by two five-minute washes and one 15-minute wash in tris-buffered saline/0.1% tween 20. Secondary antibodies were diluted in blocking buffer and incubated with the membranes for 45 minutes at room temperature. Washes were repeated as above and a final 15-minute wash was done with tris-buffered saline. ECL (Amersham, Buckinghamshire, England) was performed according to the manufacturer's instructions and autoradiographs were made.

RNA Preparation and RT-PCR

Total RNA obtained as described in Example 1. RT reactions were performed as described in Example 1. The additional primers used were: Notch3 forward 5′-GTGTGTGTCAATGGCTGGAC-3′ (SEQ ID NO:13); reverse 5′-CGATAGAGCACTCGTCCACA-3′ (SEQ ID NO:14); Notch-4 forward 5′-GGCTTCTACTCCGCTTCCTT-3′ (SEQ ID NO:15); reverse 5′-CAACTTCTGCCTTTGGCTTC-3′ (SEQ ID NO:16). Thirty-five cycles of amplification were done for each gene. Each three-step cycle was 94° for 45 seconds, 60° for 45 seconds and 72° for 30 seconds.

Cell Culture

The cells were cultured essentially as described in Example 1.

Some cultures were adapted to grow on Matrigel in MEF conditioned medium. In this case, growth medium was exposed to MEF feeder layers overnight and this medium (CM) was supplemented with fresh bFGF and beta-mercaptoethanol before feeding hES cultures. Passaging conditions were the same as described above. Growth Factor reduced Matrigel (BD Biosciences, Inc.) was diluted 1:30 in DMEM/F12 medium and applied to cell culture dishes. It was aspirated before cells were plated at a density of 50,000 cells/cm². Cells were analyzed for viability and counted on a Guava personal cytometer (Guava Technologies, Inc.).

Magnetic Sorting

Magnetic sorting was performed as described in Example 1.

EB Formation

Embryoid bodies were formed from cultures that had been growing on Matrigel and exposed to the different treatments. These cultures were exposed to Collagenase type IV or Dispase (Gibco) for 10 minutes at 37° C. until a monolayer of cells lifted off of the dish. The monolayer was passed through a pipette tip until it was broken down to small clumps of approximately 500-1000 cells. These clumps were washed in 15% FBS/1% MEM nonessential amino acids/1 mM L-glutamine/penicillin (0.5 U/ml) and streptomycin (0.5 U/ml) twice by allowing them to settle by gravity, removing the supernatant, and resuspending the loose pellet. The cells were plated onto agarose coated Petri dishes in the same medium used for washing and allowed to grow for up to 10 days.

Flow Cytometry

Cultures were harvested by Collagenase/trypsin passaging methods described above as an essentially single cell suspension, washed in 1×PBS, and fixed in 1% paraformaldehyde for 25 minutes on ice. The cells were washed in 1×PBS, blocked in 3% goat serum, and stained with primary antibodies for 30 minutes. to overnight at 4° C. The cells were washed in 1×PBS three times and stained with Secondary FITC or PE conjugated antibodies (Jackson Immunochemicals, Inc.) for 1 hour to overnight at 4° C. The cells were washed with 1×PBS three times and analyzed on a CyAn flow cytometer (DakoCytomation, Inc.).

Real Time RT-PCR

Hes1 induction by EDTA treatment was monitored by real-time RT-PCR. Cultures of bES cells on Matrigel-coated 24-well plates were treated with either 0.5% DMSO or 50 μM DAPT in CM for 4 hours to overnight before induction by EDTA exposure. The wells were exposed to 2 mM EDTA in CM (with DMSO or DAPT) for 15 minutes at 37° C. EDTA was neutralized by addition of 4.9 mM CaCl₂ and incubated for a further 1.5 hours. The cells were harvested with Trizol and RNA was extracted as described above. Real-time PCR was performed using TaqMan primers and probes (Applied Biosystems, Inc.) for Hes1 (primer sequences: Hes1-F: 5′-CTACCCCAGCCAGTGTCAAC-3′ (SEQ ID NO: 17); Hes1-R: 5′-TCAGCTGGCTCAGACTTTCA-3′ (SEQ ID NO:18); probe Hes1-P: 6FAM-CGACACCGGATAAACCAAAGACAGC-TAMARA (SEQ ID NO:19)) and normalized to GAPDH (kit from Applied Biosystems, Inc; cat. no. 402869). Reactions were run and monitored on an ABI7700 Sequence Detection System (Applied Bioysytems, Inc). Data was analyzed as described in Pfaffl et al., 2002 Nucleic Acids Research 30(9): E36. REST-XL (version 2) software was used to determine relative quantification of Hes1 gene expression. The data were expressed as the ratio of GAPDH normalized Hes1 expression of EDTA-treated to EDTA-untreated samples. DAPT treated cultures were compared to DMSO treated cultures. These values were log transformed for statistical analysis by t-test and graphed in Excel.

Results

Embryonic stem cell lines have been derived from the ICM of human blastocysts. These lines seem to require cell-cell interactions to be stabilized in an undifferentiated state. The only successful passaging techniques reported to date maintain cell-cell contact by not breaking clumps of hES cells down to single cells. Both enzymatic and non-enzymatic passaging techniques have been used herein and elsewhere to passage hES cells. The non-enzymatic techniques employ fine glass needles and pipettes to manually dissect and transfer small clumps of hES cells. Trypsin/EDTA or a combination of Collagenase type IV and trypsin/EDTA is used in the enzymatic techniques to break hES cells down to an essentially single cell suspension for transfer to fresh dishes.

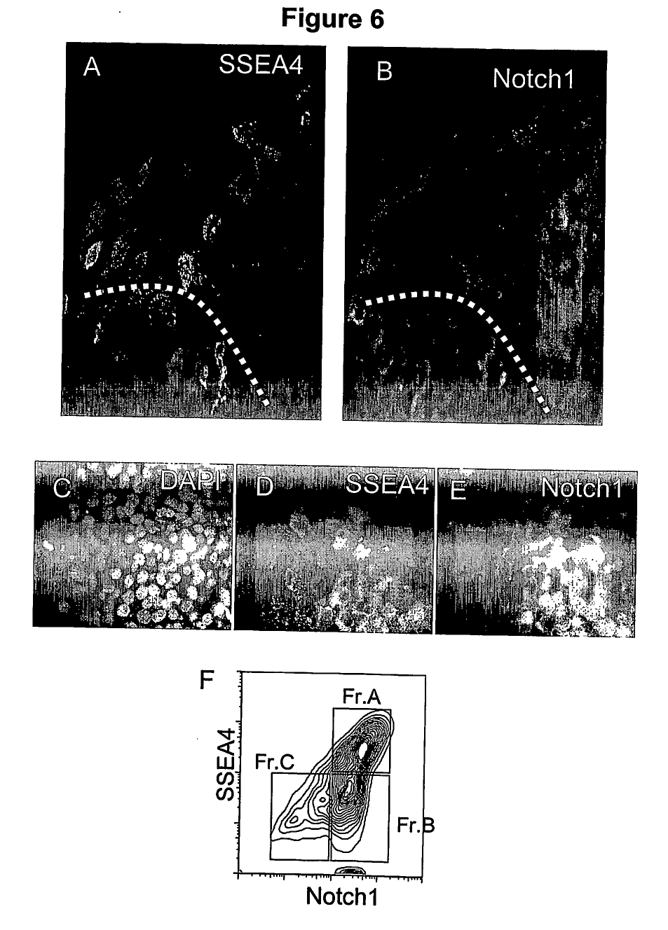

However, the passaging techniques used to date to maintain human embryonic stem cells (hES cells) resulted in heterogeneous cultures as assayed by pluripotency markers and cellular morphology. An example is shown in FIGS. 6A-F. Areas of a manually passaged colony shown below the dashed lines were morphologically unpolarized and expressed SSEA4 indicating their undifferentiated state. However, as shown in areas above the dashed line, cells in the same colony were beginning to differentiate. This was indicated by an elongated or polarized morphology. In addition, cells in this area were shutting off SSEA4 expression, first recognized as the SSEA4 epitope was gathered into endocytic vesicles. Also shown is Notch1 staining for the same area that was stained for SSEA4 (FIG. 6B). Undifferentiated cells (below dashed line) expressed high levels of Notch1. Notch1 expression was downregulated in differentiating areas in an overlapping but not identical pattern. SSEA4 positive cells in this area seemed to be a subset of the Notch1 expressing cells such that there were Notch1 positive cells that were not SSEA4 positive. This differentiation pattern was also seen in hES cultures adapted to grow on Matrigel in MEF feeder conditioned medium (see FIGS. 6C-E). SSEA4 positive cells were seen to be a subset of Notch1 expressing cells. FIG. 6F shows a two-dimensional plot of SSEA4 and Notch1 expression on individual cells from flow cytometry analysis of a manually passaged culture that had been plated on an untreated tissue culture plastic surface to allow for random differentiation. Cells fell into three general fractions by this analysis: the SSEA4high/Notch1high flow fraction (Fr. A); the SSEA4low/neg/Notch1positive fraction (Fr. B); and the SSEA4low/neg/Notch1low/neg fraction (Fr. C). This figure shows progression of marker expression upon differentiation and points out that cells from both the manual and enzymatic culture systems are heterogenous.

### Example 3

Expression of Notch Family Members and the Gamma-Secretase Complex in HESCs

Notch signaling that is mediated by a gamma-secretase mediated cleavage has previously been shown to control differentiation and proliferation in many developmental contexts. Here it is shown that hES cells express Notch-1, -2, and -3, and active forms of components of the gamma-secretase complex.

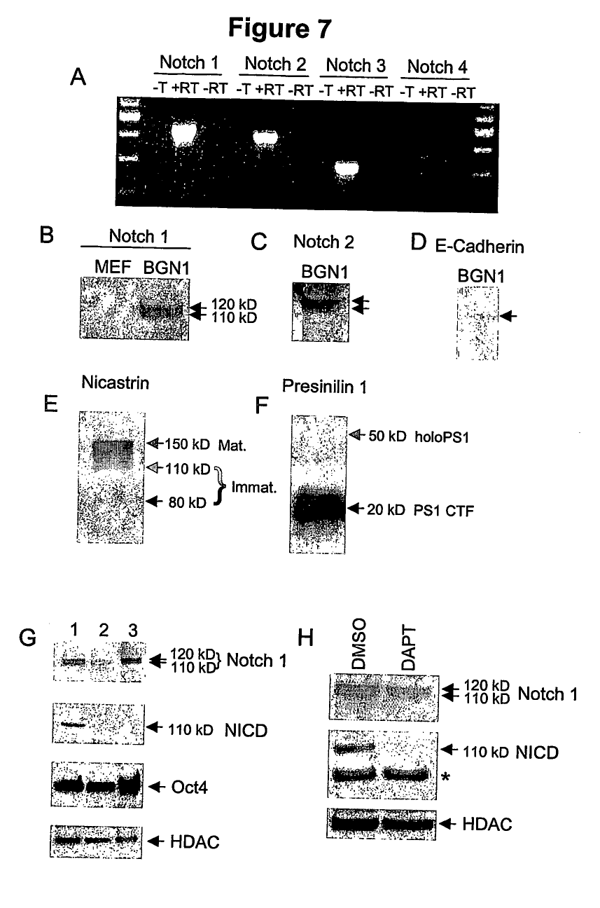

FIG. 7A shows RT-PCR analysis indicating strong expression of Notch-1, -2, and -3 in HESCs. Notch-4 was only weakly detected. In a separate set of experiments, Notch-4 was not detected. Lack of expression of Notch-4 was verified using an independent set of PCR primers. Protein for Notch-1, and -2 was detected by Western blotting using three different antibodies specific for Notch-1 and one antibody specific for Notch-2 in both BG01 and BG02 cell lines. FIG. 7B-F shows an example of these blots. The Western blot for Notch1 shown in FIG. 7B used an antibody that recognizes an epitope on the cytosolic domain. Two bands are indicated. The top ˜120 kd band detects the post-translationally processed transmembrane form of Notch1. The bottom ˜110 kd band detects the fragment of Notch1 released by gamma-secretase cleavage of the transmembrane form of Notch1. The blot shown for Notch-2 also shows this double band staining pattern resulting from gamma-secretase cleavage. In addition E-cadherin was expressed. E-cadherin can be cleaved by the gamma-secretase complex and is involved in the disruption of adherins junctions.

Undifferentiated hES cells were examined for other members of the gamma-secretase complex. Nicastrin was predominantly detected in its mature glycosylated form (Kimberly et al., 2002 J. Biol. Chem. 277:35113-35117; Kimberly et al., 2003 Proc. Natl. Acad. Sci. USA 100:6382-6387) indicated by the band migrating at ˜150 kD (FIG. 2E). Presinilin-1, which along with Presinilin-2 forms the proposed active site for gamma-secretase, was also found predominantly in it's processed mature form (FIG. 7F; Counts et al., 2001 J. Neurochem. 76:679-689; Ratovitski et al., 1997 J. Biol. Chem. 272:24536). Presinilin-1 and 2 are each processed into C-terminal fragments (CTF) and an N-terminal fragment (NTF) that reassociate with each other and may form the active site of gamma-secretase (Thinakaran et al., 1996 Neuron 17: 181-190; Li et al., 2000 Nature 405:689-694; Esler et al., 2000 Nat. Cell Biol. 2:428-434). Blots for Presinilin-1 using an antibody that recognizes the loop of the CTF showed this processed fragment (PS1 hetero, FIG. 7F) was also more abundant than the uncleaved form (PS1-holo, FIG. 7F). This was indicated by a ˜20 kD CTF (C-terminal fragment) shown in FIG. 7F. The other two members of the gamma-secretase complex, Aph1 and Pen-2, have not been examined.

The gamma-secretase complex can also be activated in hES cells to cleave Notch upon EDTA exposure. FIG. 7G shows the results of two different passaging techniques that differentially expose cells to EDTA. Notch cleavage and target gene activation can be induced by treatment of a variety of Notch and gamma-secretase expressing cell lines with Ca⁺⁺ chelators (Rand et al., 2000 Mol. Cell. Biol. 20:1825-1835; Susini et al., 2001 Proc. Natl. Acad. Sci. USA 98:15067-15072). Lanes 1 and 2 were derived from cultures that were harvested by exposure to Trypsin/EDTA. Lane 3 was harvested by exposure to collagenase IV only. Only in the cultures exposed to EDTA was the ˜110 kD Notch1 band observed. An independent antibody, NICD, which recognizes the gamma-secretase cleavage site only after it has been specifically cleaved, also only detected a band in the Trypsin/EDTA exposed samples and not in the collagenase IV only exposed lane. Oct4 was expressed in all samples, indicating a possible undifferentiated state of the cultures.

To address the possibility that trypsin non-specifically generated the 110 kD Notch1 band, DAPT, a potent and specific gamma-secretase inhibitor, was used to treat the hES cells before harvesting. The cells were grown in 50 μM DAPT or the equivalent amount of DMSO (0.5%) for 3 days and harvested for protein using Trypsin/EDTA. The NICD fragment was not generated in BGN1 cultures grown in DAPT (FIG. 7H) but was generated when treated with an equivalent amount of DMSO. The asterisk in H is a non specific band found in MEF feeders alone but not hESCs alone treated in the same manner (data not shown). HDAC is shown as a loading control. Thus, an active gamma-secretase complex was present in the human ES cells and can generate NICD upon treatment with EDTA.

### Example 4

Gamma-Secretase is Activatable in hES Cells

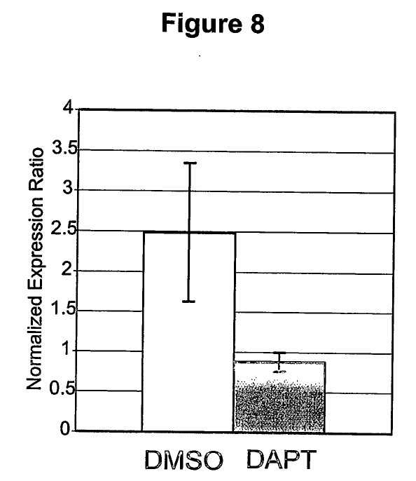

To further show that gamma-secretase is activatable in hES cells, the induction of a target gene of Notch signaling, Hes1, was assayed upon EDTA activation. FIG. 8 shows that Hes1 expression was induced by EDTA exposure but could be blocked by the addition of a gamma-secretase inhibitor, DAPT. EDTA exposure resulted in up to 10 fold induction of Hes1 in three independent experiments. This induction could be reduced to less than two-fold by the addition of DAPT in these experiments. Thus, blocking gamma-secretase blocks a target of a gamma-secretase mediated signal.

### Example 5

Inhibition of Gamma-Secretase Decreases Spontaneous Differentiation of HESCs and Stabilizes Cells in an Undifferentiated State

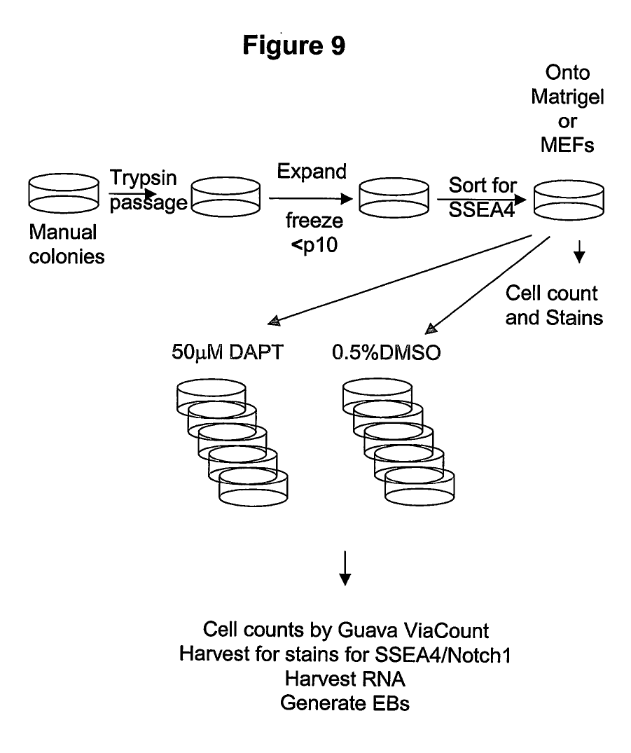

Inhibition of gamma-secretase reduces the number of spontaneously differentiated cells in the culture. Two criteria were used to show that hES cells maintain their pluripotent phenotype with inhibition of gamma-secretase. These experiments are diagramed in FIG. 9. hES cell master cultures were maintained with the manual passaging technique. A combination of Collagenase type IV treatment and Trypsin/EDTA exposure was used to passage the hES cells as single cells for no more than 10-12 passages before SSEA4 selection for early trypsin cultures, or were passaged more than 40 passages for late trypsin cultures. The cultures were selected for SSEA4 epitope expression using magnetic selection of SSEA4 antibody-stained cells. The retained fraction from the sort was plated and subjected to the treatment conditions. In some instances, SSEA4 selected cells were expanded for one or two passages. Treatment conditions were 50 μM DAPT, or 0.5% DMSO as carrier control. In some cases, these conditions were compared to untreated cells. The treated cultures were analyzed for SSEA4 expression by flow cytometry, immunohistochemistry, and EB formation.

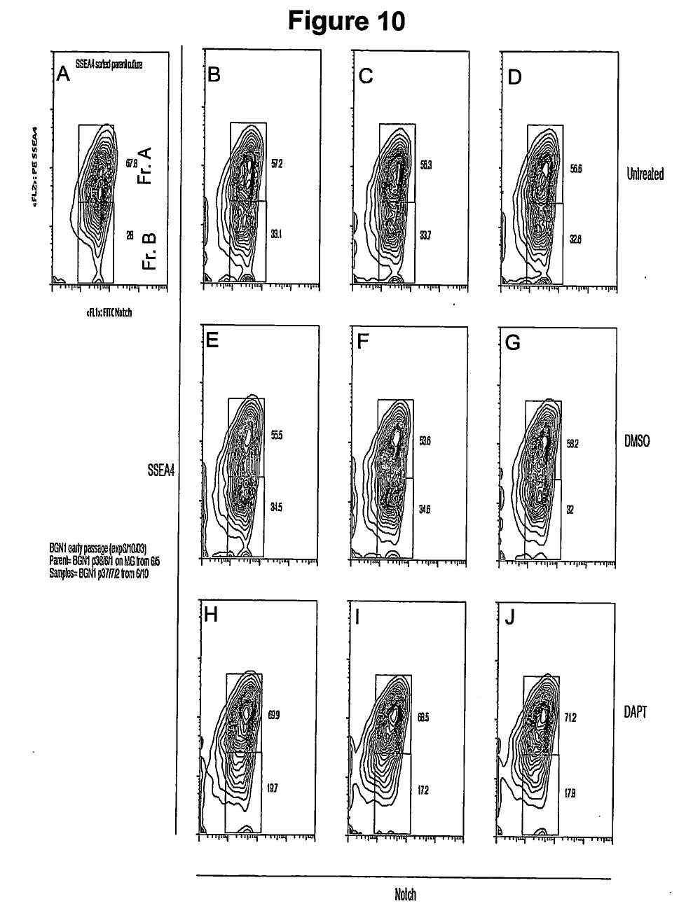

FIG. 10 shows an example of flow cytometry analysis of SSEA4 expression vs. Notch1 expression. FIG. 10A shows the parent culture of the treated cultures shown in FIGS. 5B-D. The majority of cells in the parent culture express high levels of SSEA4 and Notch1 (67.8%; Fr. A), which is indicative of the undifferentiated state of bES cells. A small portion (28%) express low levels of SSEA4 while expressing moderate levels of Notch1 (Fr. B). With DAPT treatment, the proportion of cells in the culture expressing low levels of SSEA4 is decreased and the proportion expressing high levels of SSEA4 is maintained or slightly increased compared to parent cultures and DMSO or untreated cultures (FIGS. 10H-J). In addition, when compared to the parent culture, the DMSO (FIGS. 10E-G) and untreated cultures (FIGS. 10B-D) showed a decreased proportion in Fr. A.

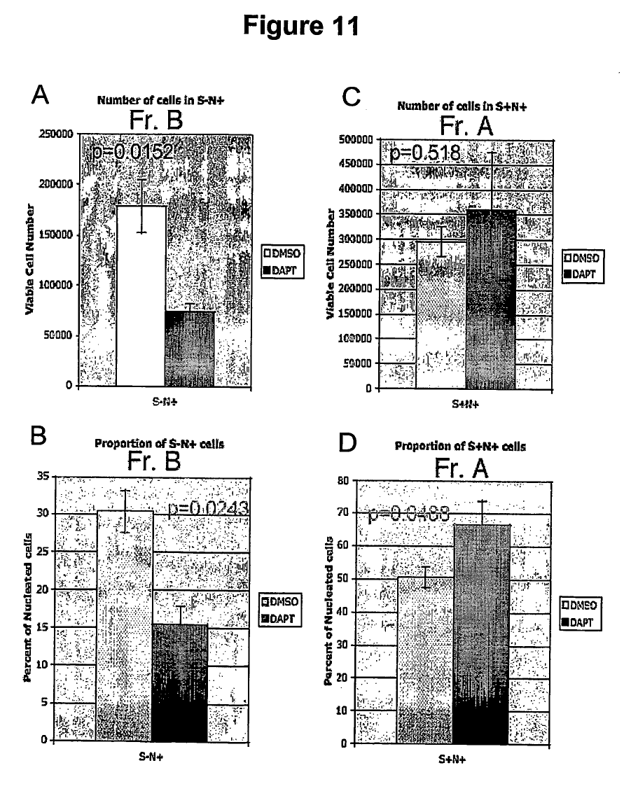

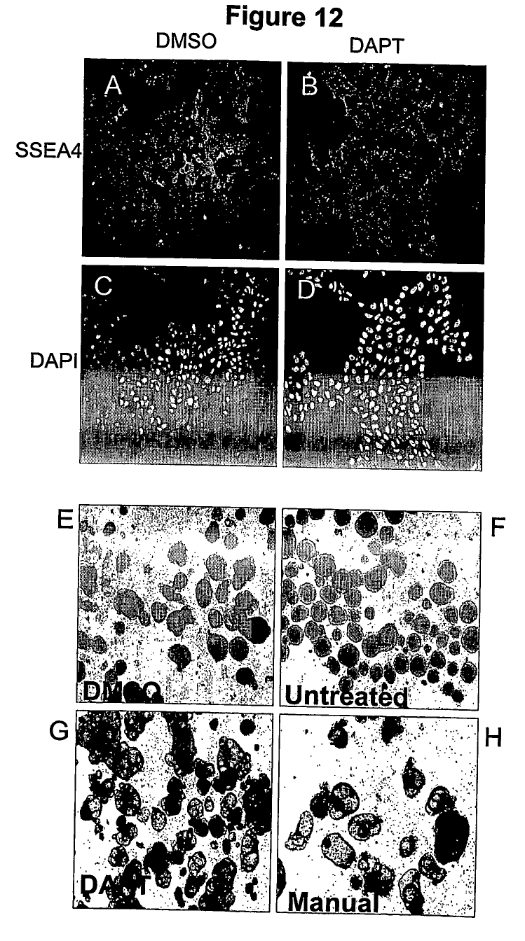

Summary data for four experiments and covering two cell lines (BGN1 and BGN2) is shown as graphs in FIGS. 11A-D. While an average of 30% of cells were in Fr. B in the DMSO treated cultures, the number of cells in Fr. B was reduced to 15% with inhibitor treatment (p=0.0243; paired t-test; FIG. 11B). There was a small but significant increase in the proportion of cells in Fr. A in the presence of DAPT (FIG. 11D). Therefore, inhibition of gamma-secretase appeared to reduce the number and proportion of differentiating cells in hES cultures leading to an increase in homogeneity of the cultures. This is further shown in immunohistochemical stains of DAPT and DMSO treated cultures. FIGS. 12A and B show SSEA4 staining of the DAPT and DMSO treated cultures, while FIGS. 7C and 7D show DAPI staining of the cultures. It can be seen that the DAPT treated cultures are more homogenous in their expression of SSEA4 (FIG. 12B vs. 12A). Further, the number of differentiated SSEA4 low or negative cells is very low compared to DMSO treated cultures. Thus, inhibition of gamma-secretase dependent signaling maintains hES cells in an undifferentiated state under these passaging conditions even when parallel untreated or DMSO treated cultures were not maintained in an undifferentiated state.

The morphology of embryoid bodies (EBs) generated from late trypsin passaged cultures maintained in DAPT vs. DMSO vs. untreated conditions suggests that inhibition of gamma-secretase can stabilize the undifferentiated state of hES cells. FIGS. 12E-H show this analysis. Note that the morphology of manual-derived EBs are cystic (FIG. 12H), whereas the untreated trypsin passage-derived EBs are not (FIG. 12F). DAPT treatment of trypsin passaged cultures returned the EBs to a cystic morphology resembling the manual-derived EBs (FIG. 12G). Thus, inhibition of gamma-secretase mediated Notch signaling or other gamma-secretase mediated signaling may improve the homogeneity of hES cell cultures. It may also suggest a molecular mechanism that controls early human development.

1. A cell culture composition comprising pluripotent cells and an inhibitor of at least one component of the gamma-secretase complex.

2. (canceled)

3. The cell culture composition of claim 1, wherein the pluripotent cells are human cells selected from the group consisting of human embryonic stem cells, human inner cell mass (ICM)/epiblast cells, human primitive ectoderm cells, and human primordial germ cells.

4. The cell culture composition of claim 3, wherein the human cells are human embryonic stem cells.

5. The cell culture composition of claim 1, wherein the inhibitor of at least one component of the gamma-secretase complex is selected from the group consisting of non-transition state analogues, transition state analogs, helical peptides containing α-aminoisobutyric acid, Fenchylamine Sulfonamide compounds, NSAIDs, and benzodiazepines.

6. The cell culture composition of claim 1, wherein the inhibitor comprises DAPT.

7. The cell culture composition of claim 1, wherein the inhibitor comprises a transition state analog selected from the group consisting of III-31-C, L-685,458, and a substrate-based difluroketone peptidomimetic.

8. The cell culture composition of claim 7, wherein the substrate-based difluroketone peptidomimetic is DFK-167.

9. The cell culture composition of claim 1, wherein the cells are stabilized in a pluripotent state for at least 10 passages.

10. The cell culture composition of claim 9, wherein the pluripotent state is determined by expression of SSEA4 and Notch1 in at least approximately 60% of the cells.

11. The cell culture composition of claim 1, wherein less than approximately 20% of the cells express HNF4alpha after approximately 10 passages.

12. The cell culture composition of claim 1, wherein the inhibitor of at least one component of the gamma-secretase complex is expressed from a feeder cell layer.

13. The cell culture composition of claim 12, wherein the feeder cell layer is genetically engineered to express the inhibitor.

14. The cell culture composition of claim 1, wherein the inhibitor of at least one component of the gamma-secretase complex inhibits Notch signaling in the pluripotent cells.

15. A cell culture composition comprising pluripotent cells and an inhibitor of Notch signaling.

16. (canceled)

17. The cell culture composition of claim 15, wherein the pluripotent cells are human cells selected from the group consisting of human embryonic stem cells, human inner cell mass (ICM)/epiblast cells, human primitive ectoderm cells, and human primordial germ cells.

18. The cell culture composition of claim 17, wherein the human cells are human embryonic stem cells.

19. The cell culture composition of claim 15, wherein the inhibitor of Notch signaling is selected from the group consisting of a gamma secretase inhibitor, and a dominant negative Notch protein.

20. The cell culture composition of claim 19, wherein the dominant negative Notch protein comprises an extracellular domain of one or more Notch proteins or a portion thereof.

21. The cell culture composition of claim 15, wherein the cells are stabilized in a pluripotent state for at least 10 passages.

22. The cell culture composition of claim 21, wherein the pluripotent state is determined by expression of SSEA4 and Notch1 in at least approximately 60% of the cells.

23. The cell culture composition of claim 15, wherein less than approximately 20% of the cells express HNF4alpha after approximately 10 passages.

24. The cell culture composition of claim 15, wherein the inhibitor of Notch signaling is expressed from a feeder cell layer.

25. The cell culture composition of claim 24, wherein the feeder cell layer is genetically engineered to express the inhibitor.

26. (canceled)

27. (canceled)

28. (canceled)

29. (canceled)

30. (canceled)

31. (canceled)

32. (canceled)

33. (canceled)

34. (canceled)

35. (canceled)

36. A method of stabilizing human pluripotent cells, comprising
a. providing a human feeder layer wherein the feeder layer expresses an inhibitor of Notch signaling, wherein the inhibitor of Notch signaling is selected from the group consisting of a gamma-secretase inhibitor, and a dominant negative Notch protein; and
b. contacting the human pluripotent cells with the human feeder layer in a culture medium
to thereby stabilize the human pluripotent cells in a pluripotent state.

37. The method of claim 36, wherein the dominant negative Notch protein comprises an extracellular domain of one or more Notch proteins or a portion thereof.

38. The method of claim 37, wherein the feeder layer is genetically engineered to express the inhibitor of Notch signaling.

39. The method of claim 36, wherein the expression of the Notch inhibitor is induced by the addition of a compound to the culture medium.

40. (canceled)

41. (canceled)

42. (canceled)

43. (canceled)

44. (canceled)

45. (canceled)

46. A method of stabilizing a pluripotent cell culture, comprising:
a. providing a pluripotent cell culture; and
b. contacting the pluripotent cell culture with an inhibitor of at least one component of the gamma-secretase complex
to thereby stabilize the pluripotent cell culture.

47. (canceled)

48. The method of claim 46, wherein the pluripotent cells are human cells selected from the group consisting of human embryonic stem cells, human inner cell mass (ICM)/epiblast cells, human primitive ectoderm cells, and human primordial germ cells.

49. The method of claim 48, wherein the human cells are human embryonic stem cells.

50. The method of claim 46, wherein the inhibitor of at least one component of the gamma-secretase complex is selected from the group consisting of non-transition state analogues, transition state analogs, helical peptides containing α-aminoisobutyric acid, Fenchylamine Sulfonamide compounds, NSAIDs, and benzodiazepines.

51. The method of claim 50, wherein the inhibitor comprises DAPT.

52. The method of claim 50, wherein the inhibitor comprises a transition state analog selected from the group consisting of III-31-C, L-685,458, and a substrate-based difluroketone peptidomimetic.

53. The method of claim 52, wherein the substrate-based difluroketone peptidomimetic is DFK-167.

54. The method of claim 50, wherein the inhibitor comprises DAPT.

55. The method of claim 46, wherein the cells are stabilized in a pluripotent state for at least 10 passages.

56. The method of claim 55, wherein the pluripotent state is determined by expression of SSEA4 and Notch1 in at least approximately 60% of the cells.

57. The method of claim 46, wherein less than approximately 20% of the cells express HNF4alpha after approximately 10 passages.

58. The method of claim 46, wherein the inhibitor is expressed from a feeder cell layer.

59. The method of claim 58, wherein the feeder cell layer is genetically engineered to express the inhibitor.

60. The method of claim 46, wherein the inhibitor of at least one component of the gamma-secretase complex inhibits Notch signaling in the pluripotent cells.
